(19) United States
(12) Patent Application Publication
(10) Pub. No.: US 2026/0152810 A1
(43) Pub. Date: Jun. 4, 2026

(54) DEVICES AND METHODS FOR RAPID POINT-OF-USE DIAGNOSTIC ASSAY

(71) Applicant: HYPERCELL TECHNOLOGIES, Ithaca, NY (US)

(72) Inventors: Bruno Jactel, Atlanta, GA (US); Loic DeBlais, Ithaca, NY (US); Hayden Bartesch, Ithaca, NY (US)

(73) Assignee: HYPERCELL TECHNOLOGIES, Ithaca, NY (US)

(21) Appl. No.: 19/388,697

(22) Filed: Nov. 13, 2025

Related U.S. Application Data
(60) Provisional application No. 63/719,773, filed on Nov. 13, 2024.
(60) Provisional application No. 63/877,919, filed on Sep. 8, 2025.

(51) Int. Cl. C12Q 1/689; C12Q 1/24; C12Q 1/6806; C12Q 1/686; G01N 35/00; G01N 35/04; G01N 35/10

(52) U.S. Cl. CPC C12Q 1/689; C12Q 1/24; C12Q 1/6806; C12Q 1/686; G01N 35/1097; G01N 2035/00475; G01N 2035/0474

(57) ABSTRACT

Disclosed are devices and methods for the rapid concentration and detection of microorganisms and microobjects in a field sample and uses thereof.

### CROSS-REFERENCE TO RELATED APPLICATIONS

This application claims the benefit of priority to U.S. Provisional Application No. 63/719,773, filed on Nov. 13, 2024, and U.S. Provisional Application No. 63/877,919, filed on Sep. 8, 2025, the entire contents of each of which are incorporated herein by reference in their entirety.

### FIELD

This invention relates generally to devices and methods for the rapid concentration and detection of microorganisms and microobjects in a field sample and uses thereof.

### BACKGROUND

Over the last decade, the direct cost of infectious diseases in animals has been estimated at more than $20 billion and indirect losses at over $200 billion to affected economies as a whole (Barratt Alyson S., et al., Frontiers in Veterinary Science, DOI 10.3389 (2019)). The impact of infections from animals spreading to humans is also staggering: WHO estimated that 11 major food borne diseases every year affect 48.4 million people and cause 59,724 deaths annually resulting in 8.78 million Disability Adjusted Life Years (DALYs) (Torgerson, Paul R et al., PLOS medicine, 12:12 e1001920 (2015)).

In the past several years, the development and application of molecular diagnostic techniques have initiated a revolution in diagnosing and monitoring infectious diseases. However, most of these techniques have been developed using expensive, sophisticated lab equipment (e.g., thermocyclers with laser detection devices for polymerase chain reaction (PCR) technology) and requiring highly skilled operators in a centralized laboratory, which is expensive and time consuming. While highly sensitive and specific, the available tests take too long to facilitate quick action to reduce the food chain contamination and quarantine animals to prevent spread. Because of the high cost of these tests, their routine use has been limited to testing only for high risk (e.g., deadly) or economically important pathogens. The tests are too slow and expensive to be widely implemented to test for pathogen contamination in multiple points of observation in the food chain, in complex manufacturing plants, or in farm animal production facilities.

For example, there are no testing methods quick enough to detect Salmonella-positive pigs upon arrival at the meat-packing plant. If a quick test were available, it would allow segregation of positive animals for end-of-the day processing to prevent cross-contamination of meat from healthy pigs. Due to testing costs and timing, detection of pathogen contamination of meats is limited to final product testing that is used to determine eligibility for sale. Test technology limitations, therefore, are driving a reactive rather than a proactive approach to prevention of contamination of foods.

The lack of fast and cost-effective methods to detect pathogen contamination in fresh fruits and vegetables is even more costly, because of the short shelf-life for many of these products. In addition, exports of food products between countries are often delayed by slow tests to detect genetically-modified or incorrectly labeled food products. Therefore, there is a need for rapid and cost-effective tests to detect genetic material, such as pathogen contamination, in foods. Across all phases of food production, early detection of pathogenic contamination through rapid diagnostic detection minimizes risks of human disease, and associated recalls that damage brand image and reduce profitability.

It should be noted that any manufacturing processes that rely on biological materials could benefit from the use of a rapid test for the detection of genes of interest. For example, pharmaceutical manufacturing could benefit from earlier detection of microbial contamination. Vaccine manufacturing could confirm product identity and freedom from adventitious agents at much earlier stages of production.

Loop-mediated isothermal amplification (LAMP) is a rapid signal amplification method for the detection of DNA or RNA targets. LAMP requires exposure of pathogenic DNA to the LAMP primers and enzymes to facilitate amplification. Typical methods involve the use of commercial DNA extraction kits to generate purified DNA samples from various pathogens and sample types. These kits typically require 10 or more steps and at least 1 hour to complete the nucleic acid purification process. Use of these kits incurs additional cost and processing time, and requires additional expensive equipment like centrifuges, tissue homogenizers, vortex mixers, and pipettes. Current commercial DNA extraction kits also use hazardous chemicals including but not limited to isopropanol, ethanol, chloroform, mercaptoethanol, and guanidine, requiring use of specific personal protective equipment, workstations, storage, and disposal infrastructures. Furthermore, current methods for sample preparation are complex, equipment-intensive and lengthy, lengthening the timeline for microbial testing. Sample preparation typically includes the extraction, purification, and processing of a biological sample to be tested (e.g. water, meat, swabs, etc.) so that the DNA or RNA of the pathogen can be detected using molecular identification techniques. The sample preparation step can take many hours, especially if enrichment is needed. The invention disclosed herein describes a breakthrough in microbial testing combining unmatched speed, accuracy, and specificity to meet the toughest demands of production, improve efficiencies, avoid use of dangerous chemicals, and shorten decision making timelines. The present application discloses methods and devices for rapid sample preparation and analysis.

### SUMMARY

In accordance with the purpose(s) of this invention, as embodied and broadly described herein, this invention, in one aspect, relates to a filtration system for capturing targeted microobjects from a sample, the system comprising: a) a first filtration module configured to capture particles at least twice as large as the targeted microobjects; b) a second filtration module downstream of and in fluid communication with the first filtration module, configured to capture the targeted microobjects from the sample; c) an injector for injecting the sample, configured to be connected upstream and in fluid communication with the first filtration module; and d) a second injector configured to be connected upstream of and in fluid communication with the first filtration module, wherein the second injector is configured to resuspend the captured microobjects in a resuspension solution, and wherein the resuspension solution comprises a buffer solution.

In some embodiments, the system further comprises a flow regulator in fluid connection with and configured between the first filtration module and the second filtration module, wherein flow regulator comprises: a) an upper connector configured to connect the flow regulator to the first filtration module; b) a lower connector configured to connect the flow regulator to the second filtration module; c) a side channel connector configured to connect the second injector to the flow regulator; and d) a 3-way valve configured to change orientation to direct flow independently, the 3-way valve having at least a first flow orientation and a second flow orientation, wherein the first flow orientation is configured to seal the side channel connector and produce fluid communication between the upper connector and the lower connector, wherein the second flow orientation is configured to seal the upper connector and produce fluid communication between the side channel connector and the lower connector, and wherein the orientation of the 3-way valve is controlled by a rotating handle.

In some embodiments, the system further comprises a cylindrical shell comprising a top end, a bottom end, and an outer wall, configured to encompass the first filtration module, flow regulator, and second filtration module, the cylindrical shell comprising: a) a first injector opening in the top end configured to provide access to the first filtration module; b) a second injector opening in the outer wall which provides access to the side channel connector; c) a turning knob opening in the outer wall comprising a turning knob operably connected with the rotating handle, wherein the turning knob opening is configured to change the orientation of the 3-way valve; and d) a waste chamber, wherein the waste chamber is located downstream of the second filtration module and within the shell; wherein the shell is composed of materials selected from the group comprising polylactic acid (PLA), polyethylene terephthalate glycol (PETG), thermoplastic polyurethane (TPU), nylon, or other 3D printed filaments, wood- or water-based filaments, acrylonitrile butadiene styrene (ABS), polypropylene (PP), polyethylene (PE) polystyrene (PS), polycarbonate (PC), or other plastic injection molding polymers, polypropylene resin, waterproof paper tubing, including 2 mm cardboard covered with 157G coating, and aluminum, and wherein the shell protects the encompassed items and prevents spilling.

In some embodiments, the first filtration module further comprises a filter cartridge encompassing one or more layers of one or more filters selected from a group comprising polyester mesh, nitrocellulose, nylon, cellulose acetate, polyether sulphone (PES), cellulose nitrate (collodion), cellulose acetate (CA), mixed cellulose esters (MCE), other cellulose membrane filters, polycarbonate, polyethylene terephthalate (PET), polyvinylidene fluoride (PVDF), ceramic membranes, polypropylene (PP), polycarbonate (PC), polytetrafluoroethylene (PTFE), and polyester (PETE), and wherein the one or more filters has a pore diameter between 0.22 and 300 μm and a filter diameter between 4 and 33 mm. In some embodiments, the second filtration module further comprises a filter cartridge encompassing one or more layers of one or more filter membranes selected from the list comprising nitrocellulose, nylon, cellulose acetate, polyether sulphone (PES), cellulose nitrate (collodion), other cellulose membrane filters, polycarbonate, polyethylene terephthalate (PET), polyvinylidene fluoride (PVDF), ceramic membranes, polypropylene (PP), polytetrafluoroethylene (PTFE), and polyester (PETE), and wherein the one or more filter membranes has a pore diameter between 0.05 and 20 μm and a filter diameter between 4 and 33 mm.

In some embodiments, the injector is selected from the group comprising a syringe; an air line; a fluid line comprising one or more of irrigation water, fermentation input including feed lines and buffers, and outputs, including harvest outputs; a line with a positive displacement pump, comprising reciprocating pumps, rotary pumps, and peristaltic pumps; a line with any centrifugal pump, comprising radial flow pumps, axial flow pumps, and mixed flow pumps; and a line with a pump selected from a group comprising submersible pumps, jet pumps, hand pumps, sump pumps, and sewage pumps.

In some embodiments, the waste chamber contains pre-loaded absorbent material capable of absorbing liquid to transform liquid waste into semi-solid waste to prevent leakage, the pre-loaded absorbent material selected from a list comprising viscose, cotton, bamboo fiber, and polyester. In some embodiments, the pre-loaded absorbent material is pre-treated with antimicrobial material selected from the list comprising silver ions, benzalkonium chloride, chlorhexidine, and essential oil.

In some embodiments, the second filtration module further comprises an isolated chamber comprising dried isothermal reagents and primers configured to release the dried isothermal reagents and primers into the second filtration module.

In some embodiments, the buffer solution comprises water, 0.1-100 mM Tris, 0.1-100 mM EDTA, 0.005-0.02% trehalose, and 0.002-2% tween.

In some embodiments, the injector further comprises an injector adapter fitted in and removable from the injector and one or more pre-loaded solvents, wherein the injector adapter is a conical tube comprising: a) an upper opening configured to accept the sample, wherein the sample is a swab; b) a lower opening; c) an outer surface comprising an O-ring configured to prevent leaking between the outer surface and the injector; d) an inner surface comprising a plurality of teeth configured to agitate the swab to suspend the targeted microobjects; and e) one or more aeration holes through the conical tube configured to optimize liquid and air transfer to prevent pressurization and depressurization of the adapter; wherein the one or more pre-loaded solvents is selected from a list comprising buffered peptone water, phosphate buffered salt, neutralized buffered peptone water (nBPW), Letheen broth, universal transport media (UTM), Nutrient Broth (NB), Luria Bertani (LB), Tryptic Soy Broth (TSB), Brain Heart Infusion broth (BHI), and water.

In another aspect, the invention relates to an automated processing system comprising: a) one or more processing modules, each comprising: i) the system of claim 2, wherein the first filtration module further comprises an input 3-way flow regulator connected to the first filtration module, and wherein the injector is a fluid line; ii) a collection tube containing a sample, wherein the collection tube and the input 3-way flow regulator are in fluid communication by the fluid line; iii) a top motor configured to control the orientation of the input 3-way flow regulator; iv) a bottom motor configured to control the orientation of the flow regulator; b) an elution reservoir containing a buffer solution, wherein the elution reservoir is in fluid communication with the input 3-way flow regulators of the one or more processing modules; c) a module frame containing the one or more processing modules; and d) a waste collection tank configured downstream of the system of the one or more processing modules.

In some embodiments, the invention further relates to a sampling bag configured to prepare a sample for injection into the filtration system of claim 1, the sampling bag comprising: a) a flexible front and rear wall, the walls being joined together along two side edges and along a bottom edge extending between the side edges to define an internal cavity with a top opening; b) a deformable wire connected to a wall of the bag below the top opening, comprising ends extending laterally from each side of the bag, the ends of the deformable wire being adapted to be bent inwardly to close the top opening; c) an opening in the front wall comprising a removable cap configured to seal the opening and provide access to the internal cavity by the injector of claim 1 configured to eject and withdraw liquid; d) a filter configured to remove large particles prior to withdrawal of liquid from the opening; and wherein the sampling bag is configured to receive a sample and allows for easy preparation of the sample.

In another aspect, the invention relates to a method for detecting microorganisms in a sample comprising: a) concentrating microorganisms from the sample using the system of claim 1, the steps comprising: i) loading the sample into the injector; ii) injecting the sample into the system; iii) capturing microorganisms from the sample in the second filtration module; iv) resuspending the captured microorganisms in a resuspension solvent, producing a concentrated sample; v) withdrawing the concentrated sample by the second injector; b) transporting the concentrated sample to a microanalyzer; and c) detecting the microorganisms in the concentrated sample by the microanalyzer.

In some embodiments, the method further comprises releasing pre-loaded dried isothermal reagents and primers into the second filtration module after resuspension of the captured microorganisms.

In some aspects, the invention relates to a point-of-use system for detecting pathogenic microorganisms, comprising the system of claim 1 and a microanalyzer, wherein the system allows for filtration of a sample, capture of microorganisms, and analysis of microorganisms captured.

In some embodiments, the microanalyzer is selected from a group comprising an ALADDIN ANALYZER™, an EzDx WeD-1 Pro device, a Mini 8-Hole Isothermal Fluorescence PCR, a CFX96 Touch Real-Time PCR Detection System.

In some aspects, the invention relates to a kit for the detection of pathogenic microorganisms, comprising: a) the system of claim 3; b) a sterile PCR tube preloaded with isothermal reagents; and c) an apparatus for testing DNA.

In some embodiments, the kit further comprises a swab for solid samples and an injector adapter fitted in and removable from the injector and one or more pre-loaded solvents, wherein the injector adapter is a conical tube comprising: a) an upper opening configured to accept the sample, wherein the sample is a swab; b) a lower opening; c) an outer surface comprising an O-ring configured to prevent leaking between the outer surface and the injector; d) an inner surface comprising a plurality of teeth configured to agitate the swab to suspend the targeted microobjects; and e) one or more aeration holes through the conical tube configured to optimize liquid and air transfer to prevent pressurization and depressurization of the adapter; wherein the one or more pre-loaded solvents is selected from a list comprising buffered peptone water, phosphate buffered salt, neutralized buffered peptone water (nBPW), Letheen broth, universal transport media (UTM), Nutrient Broth (NB), Luria Bertani (LB), Tryptic Soy Broth (TSB), Brain Heart Infusion broth (BHI), and water.

In some embodiments, the kit further comprises a sampling bag configured to prepare a sample for injection into the filtration system of claim 1, the sampling bag comprising: a) a flexible front and rear wall, the walls being joined together along two side edges and along a bottom edge extending between the side edges to define an internal cavity with a top opening; b) a deformable wire connected to a wall of the bag below the top opening, comprising ends extending laterally from each side of the bag, the ends of the deformable wire being adapted to be bent inwardly to close the top opening; c) an opening in the front wall comprising a removable cap configured to seal the opening and provide access to the internal cavity by the injector of claim 1 configured to eject and withdraw liquid; and d) a filter configured to remove large particles prior to withdrawal of liquid from the opening; wherein the sampling bag is configured to receive a sample and allows for easy preparation of the sample.

### BRIEF DESCRIPTIONS OF THE DRAWINGS

The accompanying drawings, which are incorporated in and constitute a part of this specification, illustrate (one) several embodiment(s) of the invention and together with the description serve to explain the principles of the invention.

FIG. 1 depicts a graph showing that high quantification correlation was obtained between the AOAC EB microbiology method and Hypercell EB Core EZ data (r²=0.82). A sample was detected positive for EB using Hypercell when the fluorescent signal is recorded at a given time (time to results [TTR]).

FIG. 2 depicts a graph showing the predicted CFU counts obtained with the Hypercell EB Core EZ versus confirmed counts obtained with the AOAC EB microbiology method. Hypercell EB Core EZ counts were generated based on the linear correlation described in FIG. 1. AOAC EB microbiology counts were recorded based on plating data.

FIG. 3 depicts a graph showing that Hypercell EB Core EZ provided robust results across three operators. A sample was detected positive for EB using Hypercell when the fluorescent signal is recorded at a given time (time to results [TTR]).

FIG. 4 depicts a graph showing that high quantification correlation was obtained between the AOAC EB microbiology method and the Hypercell EB test (r²=0.90). A sample was detected positive for EB using Hypercell when the fluorescent signal is recorded at a given time (TTR).

FIG. 5 depicts a graph showing that high quantification correlation was obtained between spiking dose and Hypercell Salmonella test (r²=0.93). A sample was detected positive for Salmonella using Hypercell when the fluorescent signal was recorded at a given time (TTR).

FIG. 6 depicts a graph showing the predicted CFU counts obtained with Hypercell Salmonella test versus confirmed counts obtained with microbiology method. Hypercell Salmonella counts were generated based on the linear correlation described in FIG. 5. Microbiology counts were recorded based on plating data.

FIG. 7 depicts detection profiles across fluorescence platforms. It shows a head-to-head comparison of Hypercell isothermal detection on three commercial fluorescence readers. All reactions were spiked with ˜500 CFU of Salmonella (positive) N=5 reps per unit.

FIG. 8 depicts a 2D schematic of an embodiment of the two-module filter assembly, showing the first filtration module, second filtration module, and the 3-way flow regulator, with an injector attached at the first filtration module upper connector.

FIG. 9 depicts a 2D schematic of an embodiment of the two-module filter assembly, showing the first filtration module, second filtration module, and the 3-way flow regulator, with an injector attached at the side channel connector.

FIG. 10 depicts the inner surface of the turning knob from an isometric perspective.

FIG. 11 depicts the outer surface of the turning knob from an isometric perspective.

FIG. 12 depicts a 2D schematic of an embodiment of the two-module filter assembly, showing the shell which encompasses the first filtration module, second filtration module, and the 3-way flow regulator, including the waste chamber.

FIG. 13 depicts an outer isometric view of the two-module filter assembly, showing the shell which encompasses the first filtration module, second filtration module, the 3-way flow regulator, and the waste chamber.

FIG. 14 depicts the shell of the two-module filter assembly in an open configuration, showing the inner surface, from an isometric perspective.

FIG. 15 depicts an embodiment of the injector adapter from an outer isometric view.

FIG. 16 depicts an embodiment of the injector adapter from an isometric sectional view.

FIG. 17 depicts an embodiment of the injector, comprising a syringe, an injector adapter, and a swab, shown from an isometric sectional view.

FIG. 18 depicts a 2D schematic of the automated processing unit.

FIG. 19 depicts a 2D schematic of the automated processing unit, showing multiple processing modules.

FIGS. 20A and 20B depict schematics of syringe filter raised support motifs, showing a view from the top. The grey circle depicts the outside rim of the syringe filter housing. The black dotted lines depict the motif inside the upper and lower filter chambers of the syringe filter. The motifs are created based on the mold used during plastic injection and serve as support for the membrane during the filtration procedure. The center circle depicts the connecter connecting to the upper chamber of the syringe filter where the one or more membrane or mesh is.

FIG. 21 depicts a 2D schematic of an embodiment of the two-module filter assembly, showing the first filtration module and the second filtration module without the 3-way flow regulator, with a first injector attached at the first filtration module upper connector on the left and a second injector attached at the second filtration module upper connector on the right.

FIG. 22 depicts a front elevation of an example of a sample collection bag in accordance with this invention, having a mesh filter extending substantially halfway up the bag and an opening in the bottom half of the front wall of the bag.

FIG. 23 depicts a front elevation of an example of a sample collection bag in accordance with this invention, having an in-line filter connected to the opening in the front wall of the bag.

FIG. 24 depicts a front elevation of an example of a sample collection bag in accordance with this invention, having a mesh filter extending substantially all the way up the bag and an opening in the upper half of the front wall of the bag.

FIGS. 25A and 25B depict an embodiment of a plug, comprised of an inside member and an outside member, to be inserted into the opening of the sample collection bag. FIG. 25A shows a view of the side of the inside member facing the internal cavity of the sample collection bag. FIG. 25B shows an embodiment of the outside member containing a Luer lock, showing the external-facing side.

FIGS. 26A and 26B depict an embodiment of a plug, comprised of an inside member and an outside member, to be inserted into the opening of the sample collection bag. FIG. 26A shows a view of the side of the inside member facing the sample collection bag wall, which depicts the plug cavity with a mesh filter. FIG. 26B shows an embodiment of the outside member, showing the internal-facing side.

### DETAILED DESCRIPTION

The present disclosure may be understood more readily by reference to the following detailed description of preferred embodiments of the invention and the Examples included therein and to the Figures and their previous and following description. Herein is disclosed methods and devices for rapid sample preparation and analysis. The disclosed invention provides for accurate information in real-time to proactively intervene and manage contamination, improve process efficiency, profitability, and food safety globally. The disclosed invention is rapid and highly sensitive, providing detection as low as 10 CFU in under 60 minutes without enrichment, and as low as 1 CFU with three hours of enrichment, enabling rapid identification with a high level of sensitivity, providing more sensitive and faster than the current technology. Furthermore, the disclosed invention provides an increase in versatility of sample compatibility, with a wide range of sample types including food, surfaces, liquids, air, and others, and a wide range of volumes, up to 200 mL.

The invention described herein provides several unique features which translate into several major benefits. First, faster results allow for in-real-time decision making and immediate implementation of mitigation measures. This allows food processors to reduce losses due to in-process contamination, lower the risks of product recalls and implement continuous improvement measures. Second, better and faster results allow for more informed decisions. By knowing exactly the level of contamination, the type of microorganisms involved, and if pathogenic strains are involved, the producer is provided with actionable data to implement in real time more targeted action plans. This avoids taking unnecessary measures based on false positive or false negative results, acting on the wrong target, or making late decisions. Third, the simplicity of the tests means they can be implemented on the premises by technicians not skilled in the art of microbiology, without the requirement of specific and costly equipment, without compromising the specificity and sensitivity of the results. Fourth, the versatility of the results ensures their seamless integration into existing standard operating procedures (SOPs) and do not require any changes in existing sampling procedures. In addition, because the tests are compatible with various sample types and large volumes (up to 200 mL), they can be run at different points of the production process, minimizing workflow disruptions and providing additional data points. Lastly, the high level of specificity minimizes false positives, reducing unnecessary shutdowns, product holds, and retesting, keeping operations running smoothly.

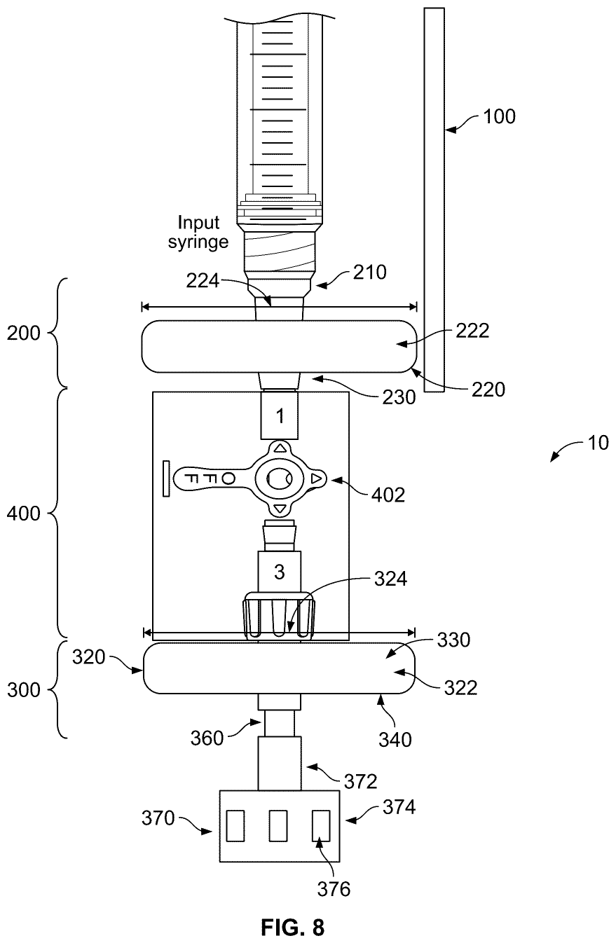

In an aspect, the present invention relates to a device for the processing and preparation of samples, comprising a two-module filter assembly 10 and an injector 100. In some embodiments, the two-module filter assembly comprises an injector 100, a first filtration module 200 downstream of the injector 100 and in fluid communication with the injector 100, configured to capture particles of a first size, a second filtration module 300 downstream of the first filtration module 200 and in fluid communication with the first filtration module 200, configured to capture particles of a second size, and a 3-way flow regulator 400 which connects the first filtration module 200 with the second filtration module 300, as depicted in FIGS. 8, 9, and 12. In some embodiments, the two-module filter assembly 10 does not include a 3-way flow regulator 400. The size of the particles captured by the first filtration module 200 is smaller than the size of the particles captured by the second filtration module 300. In some embodiments, the two-module filter assembly further comprises a cylindrical shell 500, which encompasses the first filtration module 200, second filtration module 300, and 3-way flow regulator 400, and provides access to conjoin the injector 100 and the first filtration module 200. In some embodiments, the direction of flow of a liquid through the two-module filter assembly 10 is first through the injector 100, then first filtration module 200, 3-way flow regulator 400, and second filtration module 300. In some embodiments, the 3-way flow regulator 400 may seal the first filtration module 200 from the second filtration module 300.

The disclosed invention allows for the processing of a range of samples, both liquid and solid, including but not limited to water samples, swabs, sponges, MicroTally® Swab, MicroTally® Mitts, poultry carcass rinses, fermenter broth products, and water wash tanks

### I. First Filtration Module

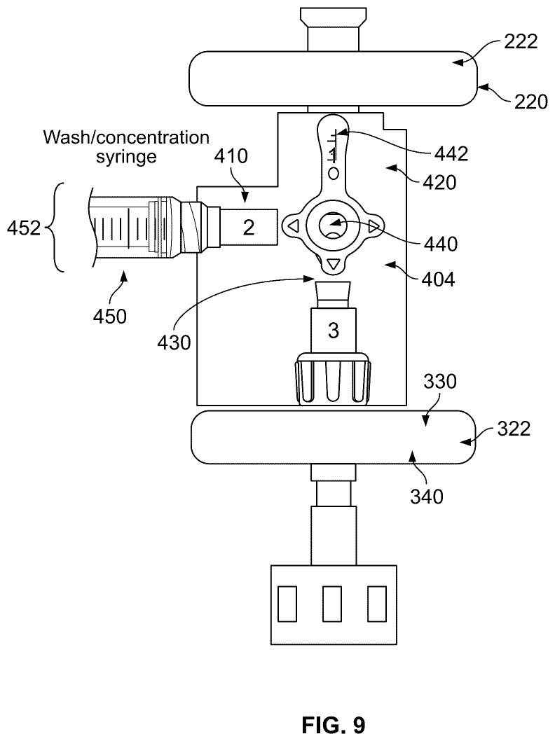

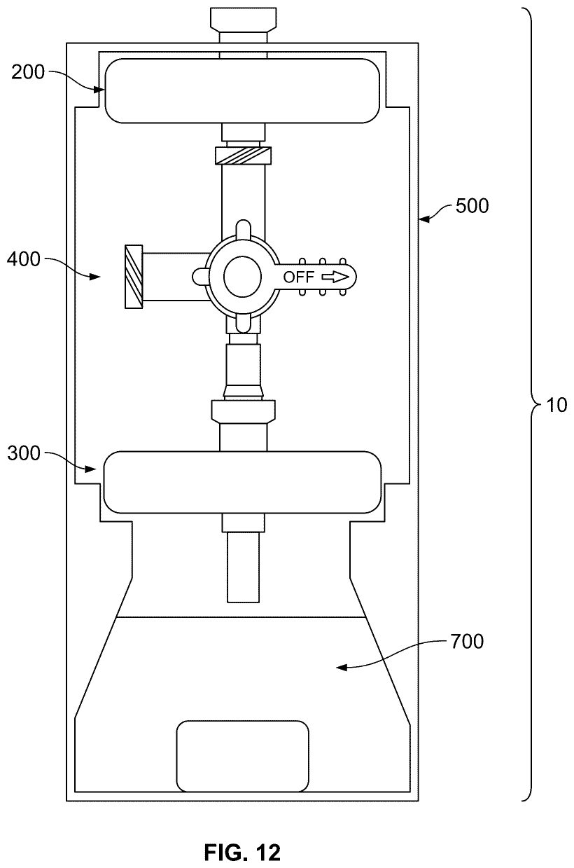

In some embodiments, the first filtration module 200 comprises a first filtration module upper connector 210 to attach an injector 100 loaded with the sample to be analyzed, wherein the injector 100 is selected from the list comprising: a first syringe, a fluid line (wherein the fluid comprises one or more of irrigation water, fermentation input including feed lines and buffers, and outputs, including harvest outputs), an air line, or any container which may be pressurized, as depicted in FIG. 8, as well as FIGS. 9 and 12. In some embodiments, the injector 100 may contain a liquid sample.

In some embodiments, the first filtration module 200 comprises a first filtration cartridge 202 containing a first filter 220 configured to pre-filter the sample to remove larger contaminants, a second filtration module 300 downstream of the first filtration module 200 comprising a second filtration cartridge 320 containing one or more second filtration membranes 322, and a 3-way flow regulator 400 which connects the first filtration module 200 and the second filtration module 300, and contains a side channel connector 410 to allow external access to the two-module filter assembly.

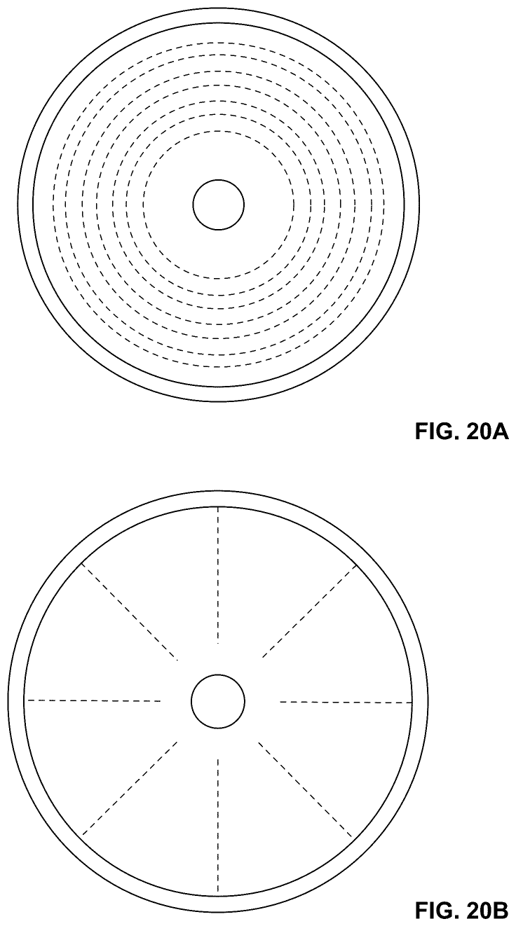

In some embodiments, the first filtration module 200 comprises a first filtration module upper connector 210, first filtration cartridge 220, and first filtration module lower connector 230, as depicted in FIG. 8. In some embodiments, the first filtration module lower connector 230 comprises a male Luer slip. The first filtration cartridge 220 contains at least one first filter 222 to remove larger contaminants, including sand, dust, meat, fat, and other larger contaminants, while allowing the passage of microorganisms and/or microobjects without yield losses. In some embodiments, the first filtration module 200 captures particles at least twice the size of the targeted microobjects. The first filtration module upper connector 210 allows rapid and easy connection with an injector 100, with a leak-proof connection. In some embodiments, the first filtration module upper connector 210 comprises a female Luer lock. The first filter module lower connector 230 can easily and tightly be attached to the 3-way flow regulator 400. In some embodiments, the first filtration cartridge 220 may be a syringe filter. In some embodiments, when the first filtration cartridge 220 is a syringe filter, multiple syringe filters may be connected in series. In some embodiments therefore, the syringe filters connected in series may have different pore sizes, diameters, and may be made of different materials. In some embodiments, the first filter 222 comprises multiple filter membranes stacked on top of each other. In some embodiments, when the first filtration cartridge 220 is a syringe filter, the syringe filter housing may be made of polypropylene (PP) and polycarbonate (PC). In some embodiments, the syringe filter housing of the first filtration cartridge 220 may contain raised support motifs 226. FIG. 20A depicts a “MAZE” raised support motif 226, while FIG. 20B depicts a “STAR” raised support motif 226. The raised support motifs 226 are situated above and below the first filter 222 within the first filtration cartridge 220, between the first filter 222 and both the first filtration module lower connector 230 and the first filtration module upper connector 210. In some preferable embodiments, the raised support motif 226 is arranged in a “MAZE” motif, as shown in FIG. 20A. In some embodiments, the first filter 222 may comprise one or more layers of polyester mesh, nitrocellulose, nylon, cellulose acetate, polyether sulphone (PES), cellulose nitrate (collodion), cellulose acetate (CA), mixed cellulose esters (MCE), other cellulose membrane filters, polycarbonate, polyethylene terephthalate (PET), polyvinylidene fluoride (PVDF), ceramic membranes, polypropylene (PP), polycarbonate (PC), polytetrafluoroethylene (PTFE), polyester (PETE) or other filter membranes. In some embodiments, the first filter 222 may be one or more non-mesh membranes, having a pore size larger than that of the second filtration membrane 322. In other embodiments, the first filter 222 may be any other filtration membrane having a pore size larger than that of the second filtration membrane 322. In some embodiments, the filter membrane 222 may be hydrophilic or hydrophobic. In some embodiments, the first filter 222 may be comprised of two or more filters or filtration membranes in sequence. In certain embodiments, the two or more filtration membranes in sequence of the first filter 222 may be two or more layers of polyester mesh. In some embodiments, the first filter 222 may comprise a polyester mesh with a pore diameter between 0.22 and 300 μm. The first filter diameter 224 may range between 4 mm and 33 mm. The first filter diameter 224 and composition may vary based on the field sample type and the volume of sample to be processed, including the turbidimetry, viscosity, and salt concentrations in the sample. In some embodiments, the pore diameter, first filter diameter 224, and type of filter or membrane may be adjusted based on the type of sample and the sample volume to be filtered. In some embodiments, the filter is between 4 and 33 mm in diameter. In some embodiments, the first filter 222 comprises a 4-33 mm PETE filter membrane. In other embodiments, the first filter 222 comprises a 4-33 mm PP filter membrane. In other embodiments, the first filter 222 comprises a 4-33 mm PTFE filter membrane. In other embodiments, the first filter 222 removes particles at least twice the size of the targeted microorganisms and/or microobjects from the sample to be analyzed. In specific embodiments, the first filter 222 removes particles>10 μm in size from the sample to be analyzed. In certain embodiments, the first filtration module 200 is a syringe filter. In some particular embodiments, the first filter 222 is a 25 mm PP filter membrane with 10 μm pore size, encapsulated in a syringe filter. In some embodiments, liquid may be passed through the first filtration module 200 by attaching the injector 100 by the first filtration module upper connector 210, and injecting liquid into the first filter cartridge 220 and through the first filter 222. In other embodiments, the injector 100 may contain air. In some such embodiments, an operator may collect air using the injector 100, which in some embodiments is a syringe. In some such embodiments, air may be passed through the first filtration module 200 by attaching the injector 100 by the first filtration module upper connector 210, and injecting air into the first filter cartridge 220 and through the first filter 222. Particles larger than the pore size of the first filter 222 cannot pass through the first filter 222 and remain on the side of the first filtration membrane closest to the injector 100. The liquid then passes through the first filter membrane lower connector 230 and into the connected 3-way flow regulator 400.

### II. 3-Way Flow Regulator

The 3-way flow regulator 400 comprises a regulator upper connector 420, a regulator lower connector 430, a 3-way valve 440, and a side channel connector 410, as depicted in FIG. 9 as well as FIGS. 8 and 12. The regulator upper connector 420, configured upstream of the 3-way valve 440 and closer to the first filtration module 200, allows a rapid and easy leak-proof connection with the first filtration module 200 through connection with the first filter module lower connector 230. The regulator lower connector 430, configured downstream of the 3-way valve 440 and closer to the second filtration module 300, allows a rapid and easy connection with the second filtration module 300 through connection with the second filter module upper connector 310. The regulator lower connector 430 and regulator upper connector 420 are configured approximately parallel and in line with one another such that liquid may pass from one to another with proper configuration of the 3-way valve 440.

The side channel connector 410 is configured approximately perpendicular to and between the regulator upper connector 420 and regulator lower connector 430. The side channel connector 410 allows a rapid and easy connection with a second injector 450.

In some embodiments, the 3-way flow regulator 400 may be configured in two orientations of the 3-way valve 440, consisting of a first flow orientation 402 and a second flow orientation 404. The orientations of the 3-way valve 440 may be controlled by a user by the rotating handle 442. The rotating handle 442 is rotated at 90° angles to achieve the three flow orientations. In the first flow orientation 402, the rotating handle 442 is turned parallel with and towards the side channel connector 410 and perpendicular the regulator upper connector 420 and regulator lower connector 430, which seals the side channel connector 410 and allows flow from the first filtration module 200 to the second filtration module 300. In some embodiments, the side channel connector 410 may comprise a female Luer lock. In the second flow orientation 404, the 3-way valve 440 is turned approximately 90° clockwise from the first flow orientation 402 to be configured parallel with and towards the regulator lower connector 430, and perpendicular the side channel connector 410, which seals the regulator upper connector 420 and allows flow from the side channel connector 410 to the second filtration module 300. In some embodiments, the regulator upper connector 420 may comprise a female Luer slip. In some embodiments, the regulator lower connector 430 may comprise a male Luer lock. FIG. 8 depicts the 3-way flow regulator 400 in the first flow orientation 402, and FIG. 9 depicts the 3-way flow regulator 400 in the second flow orientation 404.

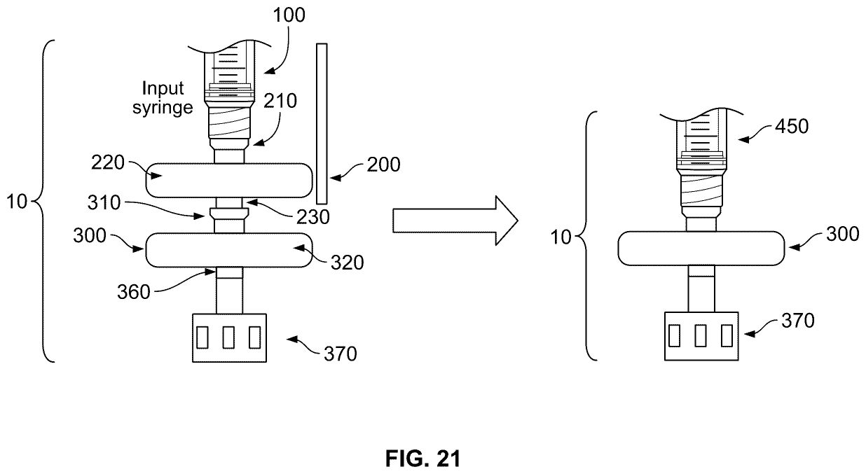

In other embodiments, the first filtration module 200 is connected directly to the second filtration module 300 by the first filter module lower connector 230 and second filtration module upper connector 310. This is depicted in FIG. 21. After dispensing the sample from the injector 100, the operator may disconnect the first filter module lower connector 230 and second filtration module upper connector 310 and connect a second injector 450.

### III. Second Filtration Module

The 3-way flow regulator 400 is configured upstream of the second filtration module 300, as depicted in FIG. 8, as well as FIGS. 9 and 12. In some embodiments, the second filtration module 300 comprises a second filtration module upper connector 310, second filter cartridge 320, and second filtration module lower connector 360. In some embodiments, the second filtration module upper connector 310 comprises a female Luer slip. In some embodiments, the second filtration module lower connector 360 comprises a male Luer slip. The second filter cartridge 320 comprises an upper chamber 330, a second filtration membrane 322, and a lower chamber 340. The upper chamber 330 is configured upstream of the second filter membrane 322, and the lower chamber 340 is configured downstream of the second filter membrane 322. The second filtration membrane 322 captures microorganisms and/or microobjects from the processed sample. The second filtration membrane 322 may comprise nitrocellulose, nylon, cellulose acetate, polyether sulphone (PES), cellulose nitrate (collodion), other cellulose membrane filters, polycarbonate, polyethylene terephthalate (PET), polyvinylidene fluoride (PVDF), ceramic membranes, polypropylene (PP), polytetrafluoroethylene (PTFE), polyester (PETE) or other filter membranes. The identity of the composition of the second filtration membrane 322 may enable different product extraction. In some embodiments, the filter membrane 322 may be hydrophilic or hydrophobic. The second filter membrane diameter 324 may range between 4 and 33 mm. The second filter membrane diameter 324 and composition may vary based on the field sample type and the volume of sample to be processed. In some embodiments, the filter is 4-33 mm in diameter. The second filtration membrane 322 may range in pore size between 0.05 and 10 μm. In some embodiments, the second filtration membrane 322 may comprise a 0.45 μm pore size. In certain embodiments, the second filtration membrane 322 is encapsulated in a syringe filter. In some embodiments, when the second filtration cartridge 320 is a syringe filter, multiple syringe filters may be connected in series. In some embodiments therefore, the syringe filters connected in series may have different pore sizes, diameters, and may be made of different materials. In some embodiments, the second filtration membrane 322 comprises multiple membranes stacked on top of each other. In some embodiments, when the second filter cartridge 320 is a syringe filter, the syringe filter housing may be made of polypropylene (PP) and polycarbonate (PC). In some embodiments, the syringe filter housing of the second filter cartridge 320 may contain raised support motifs 326. FIG. 20A depicts a “MAZE” raised support motif 326, while FIG. 20B depicts a “STAR” raised support motif 326. The raised support motifs 326 are situated both above and below the second filtration membrane 322 within the second filtration cartridge 320, between the second filtration membrane 322 and both the second filtration module lower connector 360 and the second filtration module upper connector 310. In some preferable embodiments, the raised support motif 326 is arranged in a “MAZE” motif, as shown in FIG. 20A. In other particular embodiments, the second filtration membrane 322 may comprise a 25 mm 0.45 μm hydrophobic PTFE membrane filter. In another such embodiment, the PTFE membrane filter may be encapsulated in a syringe filter. In some embodiments, the second filtration membrane 322 may be coated with an antimicrobial composition.

In some embodiments, the second filtration module 300 comprises an upper chamber 330, which the sample encounters before the second filtration membrane 322, and a lower chamber 240, which the sample encounters after the second filtration membrane 322, both contained by the second filter cartridge 320.

In some embodiments, the second filtration module 300 further comprises an end adapter 370, which attaches to the second filtration module lower connector 360. The end adapter 370 is depicted in FIG. 8. The role of the end adapter 370 is to prevent clogging and contamination of the second filtration module 300 by an adsorbent material located in the waste chamber 700, described below, which is located below the second filtration module 300. The end adapter 370 further comprises a connecter neck 372, which connects the end adapter 370 with the lower connector 360. In some embodiments, the end adapter 370 is composed of PLA. In some embodiments, the end adapter 370 further comprises an adapter container 374 comprising a plurality of container holes 376 in the material of the end adapter 370, and a mesh inside the adapter container 374 which allows liquid to pass but provides a barrier against larger objects to the second filtration module lower connector 360. In some embodiments, the dimensions of the adapter container 374 are between 1 mm by 1 mm and 3 cm by 3 cm. In some embodiments, the dimensions of the adapter container 374 are 8 mm by 8 mm. In some embodiments, the dimensions of the container holes 376 are between 0.1 mm by 0.1 mm and 15 mm by 15 mm. In some embodiments, the dimensions of the container holes 376 are 2 mm by 2 mm.

### IV. Waste Chamber

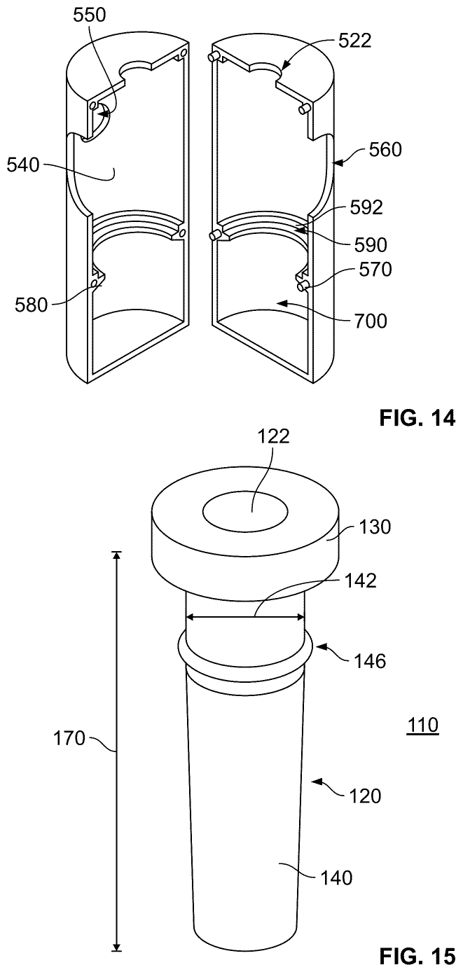

In some embodiments, a waste chamber 700 may be configured downstream from the second filtration module, as depicted in FIG. 12 and FIG. 14. Waste chamber 700 may be configured as part of the shell 500. In some embodiments, the waste chamber 700 is cylindrical. In some embodiments, the waste chamber 700 is made of the same material as the shell 500. In some embodiments, the waste chamber 700 may contain up to 23 mL of liquid. In some embodiments, the dimensions of the waste chamber 700 are 33 mm in diameter by 28 mm in height. In some embodiments, the waste chamber 600 is pre-loaded with an absorbent material capable of transforming the liquid waste into semi-solid waste. In some embodiments, the adsorbent material is a dried hydrophilic compressed towel, compressed into a shape having dimensions of 0.01 inches by 0.01 inches by 5 inches by 5 inches. In some embodiments, the shape is a cylinder. In some embodiments, the shape is a cube. In some embodiments, the absorbent material has dimensions of 0.8 inches by 0.3 inches. In particular embodiments, the absorbent material is small enough to fit within the waste chamber 700 when dry, and to expand to approximately the volume of the waste chamber 700 after having absorbed liquid waste. In some embodiments, the dried hydrophilic compressed towel has the capacity to absorb up to 25 mL of liquid within 15 seconds. In some embodiments, the towel may expand to occupy 70-80% of the total chamber volume. In some embodiments, when unfolded, the towel is a thin sheet of 8.7 by 8.7 inches. The towel may be comprised of any hydrophilic material capable of compression, including viscose, cotton, bamboo fiber, or polyester. The transformation from liquid waste to semi-solid waste prevents leakage during or after filtration.

In some embodiments, the towel is pre-treated with an antimicrobial material. In some embodiments, this antimicrobial material comprises silver ions, benzalkonium chloride, chlorhexidine, or essential oil. The antimicrobial material avoids the proliferation of microorganisms inside the waste chamber 700. The antimicrobial material allows for the safe disposal of the device without biohazard risks.

### V. Injector Adapter

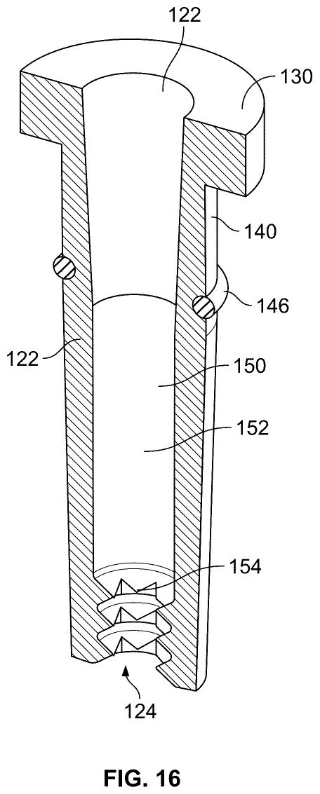

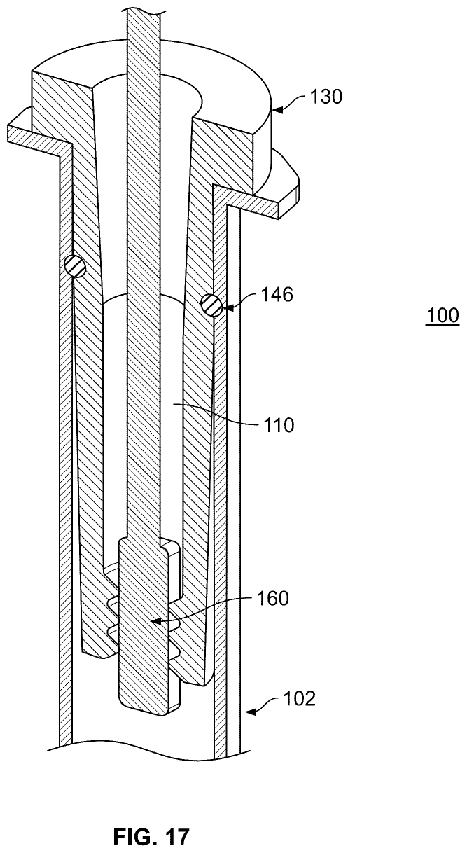

In some embodiments, the injector 100 may comprise a syringe 102 and an injector adapter 110 for suspension of environmental samples (e.g., swabs, sponges). The injector adapter 110 enables processing of swab, sponge, and otherwise solid samples directly with the disclosed invention. Thereby, sample preparation can be done with the disclosed one tool without the requirement for additional supplies (e.g., collection bag), nor pre-homogenization of the sample into an additional device, nor transfer of contaminated liquids between devices. The injector adapter 110 comprises a conical tube 120 and a collar 130, the collar 130 configured at the end of the conical tube 120 with the largest diameter, as depicted in FIGS. 15-17. The collar 130 comprises an upper opening 122, configured at the end of the collar furthest from the conical tube. The conical tube 120 comprises a lower opening 124, configured at the end of the conical tube 120 with the smallest diameter. The suspension of solid sample may occur when the injector adapter 110 with loaded swab or sponge is placed in a syringe 102 and washed with a pre-loaded solvent selected from a list comprising one or more of buffered peptone water, phosphate buffered salt, neutralized buffered peptone water (nBPW), Letheen broth, universal transport media (UTM), Nutrient Broth (NB), Luria Bertani (LB), Tryptic Soy Broth (TSB), Brain Heart Infusion broth (BHI), water, or any other culturing broth used in microbiology.

The upper opening 122 allows the insertion of a swab or sponge. In some embodiments, the upper opening 122 is closed with a rubber cap 126 to prevent introduction of contaminants and/or release of liquids. The collar 130 is a ring of material which protrudes from the outer surface 140, which is configured to rest on the opening of the syringe 102, as shown in FIG. 15, wherein the injector adapter 110 is inserted into the syringe 102. The conical tube 120 and collar 130 are configured such that the inner surface 150 of the injector adapter 110 is substantially smooth between the collar 130 and conical tube 120, without an internal lip. The inner surface 150 guides the swab or sponge to the lower opening 124. Configured at the lower opening 124, on the inner surface 150, exists a plurality of teeth 154, which both secure an environmental sample including a swab or sponge and agitate and massage the sample to release the microorganisms and other particles into the syringe 102 pre-filled with a liquid. The teeth 154 are aligned in at least two vertical rows. In some embodiments, the teeth 154 are aligned in three vertical rows. The teeth 154 are pointed serrations on the inner surface 150, pointing into the internal chamber 152 such that the swab or sponge is held snugly enough to agitate the swab or sponge with repetitive movement, but loosely enough for the swab or sponge to be easily movable. The teeth 154 extrude into the internal chamber 152 between 2 mm and 6 mm. The vertical rows of teeth 154 may comprise the lower 0.5 mm to 2 mm of the injector adapter 110, closes to the lower opening 124.

The dimensions of the injector adapter 110 may change according to the size of syringe 102 used, which may be larger or smaller depending on the amount of sample to be processed. The outer surface top diameter 152 is such that the diameter is approximately the same as the inner diameter of the syringe 102. For example, for a syringe 102 having a maximum suggested volume of 30 mL, with a diameter of 22 mm, the outer surface top diameter 142 of the injector adapter 110, measured of the outer surface 140 just under the collar 130, is approximately 22 mm, and the length 170 is approximately 90 mm. In some embodiments, the outer surface top diameter 142 may range from 12 to 28 mm. In other embodiments, the dimensions of the injector adapter 110 may be scaled from the above example to fit other types of injectors and syringes, including 5 mL, 10 mL, 15, mL, and 50 mL syringes.

Configured on the outer surface 140 of the injector adapter 110 is an O-ring 146. The O-ring 146 allows a tight fit of the injector adapter 110 into the syringe 102, limiting the risk of spills during handling. The O-ring 146 has a diameter of sufficient magnitude as to accommodate the outer surface 140, and to provide a seal with the internal surface of the syringe 102. The diameter of the O-ring 146 may change according to the size of syringe 102 and injector adapter 110 used. The diameter of the O-ring 146 may range from 12 mm to 28 mm. In some embodiments, this dimension is the same as the outer surface top diameter 142. The O-ring 146 may be configured on the outer surface 140, between 3 mm and 20 mm from the collar 130.

The present disclosure also relates to aeration holes 144 positioned within the conical tube 120 of the injector adapter 110. Aeration holes 144 run through the thickness 122 of the conical tube 120 between the outer surface 140 and inner surface 150. Aeration holes 144 are configured to optimize the liquid/air transfer between the internal chamber 152 and the interior of the syringe 102. Thus, the aeration holes 144 facilitate release of microorganisms and/or microobjects collected by the swab or sponge into the liquid. The aeration holes 144 also avoid creating pressurized and depressurized conditions during handling of the device and insertion of the swab or sponge. One, two, three, or more aeration holes 144 may be positioned along the length 170. Aeration holes 144 include outer apertures 146, positioned on the outer surface 140 and inner apertures 148, positioned on the inner surface 150. The diameter of the aeration holes 144 may range between 2 and 5 mm. The aeration holes 144 are positioned between the O-ring 146 and the lower opening 124.

### VI. Shell

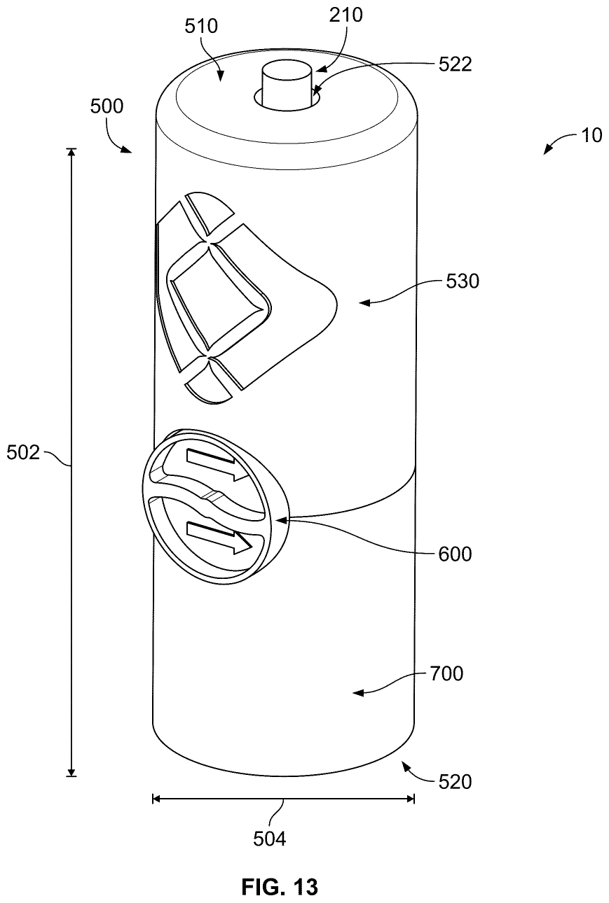

In some embodiments, the two-module filter assembly further comprises a shell 500, which encompasses the first filtration module 200, second filtration module 300, and 3-way flow regulator 400, and provides access to conjoin the injector 100 and the first filtration module 200, as depicted in FIGS. 13, 14, 18, 19, 20, and 21, as well as FIG. 12. In some embodiments, the shell 500 is a cylinder. The shell 500 further comprises a top end 510 and bottom end 520 to provide an enclosure, limiting spills. In some embodiments, the top end 510 and bottom end 520 are both substantially flat. The shell 500 has a length 502, which may range from 0.6 cm to 20 cm. The shell 500 also has a width 504, which may range from 13 to 55 mm. In some preferable embodiments, the length 502 is 17 cm. In some preferable embodiments, the width 504 is 87 mm. The length 502 of the shell 500 is configured with the bottom 520 at approximately an internal 90-degree angle, such that the shell 500 can stand upright with the bottom 520 as the base. The length 502 of the shell 500 is configured with the top 510 at approximately 90-degree internal angle. In some embodiments, this angle may be curved. The cylindrical shell 500 further comprises an outer wall 530 and inner surface 540, which are substantially flat.

The shell 500 may be composed of materials selected from the group comprising polylactic acid (PLA), polyethylene terephthalate glycol (PETG), thermoplastic polyurethane (TPU), nylon, or other 3D printed filaments, wood- or water-based filaments, acrylonitrile butadiene styrene (ABS), polypropylene (PP), polyethylene (PE) polystyrene (PS), polycarbonate (PC), or other plastic injection molding polymers, polypropylene resin, waterproof paper tubing, including 2 mm cardboard covered with 157G coating, aluminum, or other packaging materials. The purpose of the shell 500 is to protect the mechanics of the filtration assembly inside, especially when used as a point-of-care device in field work. The shell 500 also provides ease of use, as the shell 500 will be packaged along with the other components of the two-module filter assembly 10, fully assembled. The shell 500 also allows the two-module filter assembly 10 to be ergonomically favorable. Additionally, the shell 500 prevents spilling, and thus contamination.

The shell 500 comprises a plurality of access openings, the openings strategically configured to provide access to the first filtration module 300, second filtration module 300, and 3-way flow regulator 400. The top end 510 includes a first injector opening 522, which provides access to the first filtration module 200 via the first filtration module upper connector 210. In some embodiments, the first filtration module upper connector 210 extends past the length 502 of the shell 500, and out of the first injector opening 522. This provides access to the first filtration module upper connector 210 to the injector 100. The first injector opening 522 is located in the center of the top 510, and has a diameter ranging from 11 to 15 mm. The shell 500 further comprises a second injector opening 550 in the outer wall 530, located between 19 and 30 mm down from the top 510 of the shell 500. The second injector opening 550 provides access to the side channel connector 410 of the 3-way flow regulator 400. The second injector 450 may be attached to the side channel connector 410 through the second injector opening 550, as depicted in FIG. 9 and FIG. 14. The diameter of the second injector opening 550 may range from 11 to 15 cm. Located a 90 degree turn from the second injector opening 550, the shell 500 further comprises a turning knob opening 560. The turning knob opening 560 is configured such that it provides access to the rotating handle 442 of the 3-way flow regulator 400. In some embodiments, the turning knob opening 560 is filled with a turning knob 600. The purpose of the turning knob 600 is to facilitate rotation of the rotating handle 442 while minimizing the risk of spilling of sample out of an open turning knob opening 560.

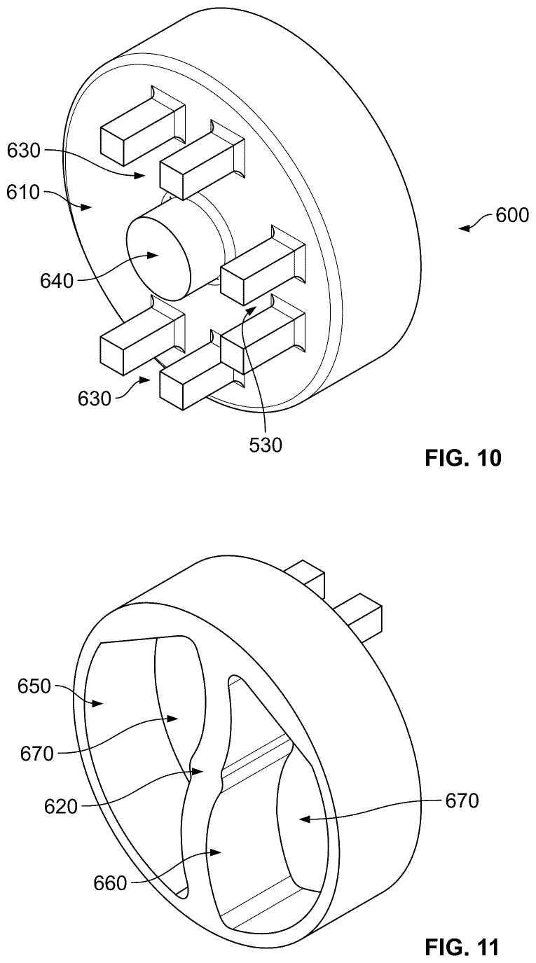

The turning knob 600 is substantially circular and configured planar and parallel to the rotating handle 442, as depicted in FIGS. 10 and 11. The turning knob 600 provides an ergonomic and easy rotation of the rotating handle 442 to change the orientation of the 3-way flow regulator 400. In some embodiments, the turning knob 600 is 3D printed. In some embodiments, the turning knob 600 may be composed of the same material as the shell 500. In some embodiments, the turning knob 600 may be composed of one or more material selected from the list comprising 3D printed filaments (e.g., Polylactic Acid [PLA]), wood- or metal-based filaments, plastic injection molding polymers (e.g., Acrylonitrile Butadiene Styrene [ABS] and polypropylene resin). The diameter of the turning knob 600 is about the same as the diameter of the turning knob opening 560. The turning knob 600 further comprises a turning knob inner surface 610. The inner surface 610 comprises three pin sets 630, which fit around the 3-way flow regulator 400, ensuring a secure and tight fit. These pins sets fit on either side of the side channel connector 410, the regulator upper connector 420, and the regulator lower connector 430. The inner surface further comprises a turning knob connector 640. In some embodiments, the turning knob connector 640 is glued to the rotating handle 442. In some embodiments, the turning knob 640 is otherwise affixed to the rotating handle 442. The outer surface 530 includes raised outer walls 650, which are flush with an internal ridge 660, which bisects the circular turning knob 600. In some embodiments, the ridge 660 is in a tangent-shaped curve, as shown in FIG. 11. In some embodiments, the ridge 660 is configured with the pin sets 630 on the inner surface 610 such that the ridge 660 bisects two pin sets 630. On either side of the ridge 660 are cavities 670. This combined structure, with outer walls 650, ridge 660, and two cavities 670, allows the user to easily rotate the turning knob 600. Rotation of the turning knob 600 causes rotation of the rotating handle 442, leading to a change in orientation of the 3-way flow regulator, as depicted in FIGS. 8-12. In some embodiments, the outer surface 620 has one or more arrow shape motifs to indicate the direction of the mechanisms, wherein the arrow shape motifs run parallel to the ridge 660. In these embodiments, the direction of the arrow indicates the direction of flow.

The shell 500 may be split open vertically to reveal its inner surface 540. In some embodiments, the vertical split occurs bisecting the turning knob opening 560 and the first injector opening 522. The inner surface 420 is substantially flat except for a support ledge 590, which comprises a ring of material extending from the inner surface 420. The distance of this extension is such that the second filter cartridge 320 rests on the side of the ledge closest to the top 510. In some embodiments, the support ledge 590 further comprises a ridge 592 configured on the side of the ledge 590 closes to the top 510. The ridge 592, extends from the inner surface 540 such that it holds the second filter cartridge 320 in place to prevent excess movement. The support ledge 590 also serves as a barrier to the waste chamber 700. The waste chamber 700, as described above, is between the support ledge 590 and the bottom 520.

In embodiments depicted in FIG. 21, wherein the first filtration module 200 is directly connected to the second filtration module 300, the shell 500 may be split in half lengthwise to allow for detachment by the operator to attach the second injector 450.

### VII. Isothermal Reagents and Primers

In some embodiments, isothermal reagents and primers 350 are preloaded into the upper chamber 330 of the second filtration module 300. In some embodiments, the isothermal reagents and primers 350 are released inside the upper chamber 330 once the filtration of the sample is complete. The isothermal reagents and primers 350 may be one or more selected from the list comprising Bst polymerase, nucleotides, primers, and co-factors. In some embodiments, the isothermal reagents and primers 350 are preloaded into the upper chamber 330 of the second filtration module 300. In some embodiments, the isothermal reagents and primers 350 are lyophilized after they are preloaded into the upper chamber 330 of the second filtration module 300. In some embodiments, the isothermal reagents and primers 350 are preloaded into an isolated chamber attached to the upper chamber 330. The lyophilized isothermal reagents and primers 350 may be released into the upper chamber 330 of the second filtration module 300 by inducing mechanical pressure on the isothermal reagent button 532, located outside and above the upper chamber 330 of the second filtration module 300. In some embodiments, the lyophilized isothermal reagents and primers 350 may be released into the upper chamber 330 in another manner. The lyophilized isothermal reagents and primers 350 are then resuspended into the sample being processed in the upper chamber 330 of the second filtration module 300. The isothermal reagents and primers 350 may provide different types of isothermal reactions. In some embodiments, the isothermal reagents and primers 350 may be used for detection and quantification, wherein the operators of the device will use the isothermal reagents to detect and quantify contaminants using fluorescent detection, in some embodiments using the ALADDIN ANALYZER™. In some embodiments, the isothermal reagents and primers 350 may be used for just detection, wherein the operators of the device will use the colorimetric isothermal reagents to detect contaminants based on a visual color change after incubation at 65° C., with no need to measure the fluorescent signal.

### VIII. Second Injector

In some embodiments, a second injector 450 containing a resuspension solvent may be attached to the side channel connector 410, as depicted in FIG. 9. In some embodiments, the second injector 450 does not contain a resuspension solvent. In some embodiments, the resuspension solvent is added to the two-module filter assembly 10 by an implement other than the second injector 450. In some embodiments, the second injector 450 is a syringe. The resuspension solvent may be selected from the group comprising one or more of water, a buffer solution, a Tris(hydroxymethyl)aminomethane (Tris) buffer solution, ethylenediaminetetraacetic acid (EDTA), Tween, Trehalose, a Tris-EDTA-Tween-Trehalose buffer solution, Triton X, bovine serum albumin (BSA), other buffer solutions of pH 7-9, and other buffer solutions. In some embodiments, the solvent may be Tris-EDTA buffer solution, a Tris-EDTA-Tween buffer solution, a Tris-EDTA-Tween-Trehalose buffer solution, a Tris-EDTA-Tween-Trehalose buffer solution optionally comprising water, 0.1-100 mM Tris, 0.1-100 mM EDTA, 0.005-0.02% trehalose, and 0.002-2% tween, other buffer solutions of pH 7-9, and other buffer solutions. In some embodiments wherein the solvent contains Triton X, the concentration of Triton X may be between 0.001% and 0.1%. In some embodiments wherein the solvent contains BSA, the concentration of BSA may be between 0.05 and 0.5%. In some embodiments, any of the previously listed buffer solutions may further comprise one or more of (2-(N-morpholino)ethanesulfonic acid) (MES), Bis-Tris, N-(2-acetamido)iminodiacetic acid (ADA), N-(2-acetamido)-2-aminoethanesulfonic acid (ACES), piperazine-N,N′-bis(2-ethanesulfonic acid) (PIPES), MOPSO, Bis-Tris Propane, N,N-Bis(2-hydroxyethyl)taurine (BES), 3-(N-morpholino)propanesulfonic acid (MOPS), 2-{[1,3-Dihydroxy-2-(hydroxymethyl)propan-2-yl]amino}ethane-1-sulfonic acid (TES), 4-(2-hydroxyethyl)-1-piperazineethanesulfonic acid (HEPES), 3-(N,N-Bis [2-hydroxyethyl]amino)-2-hydroxypropanesulfonic acid (DIPSO), 4-(N-Morpholino)butanesulfonic acid (MOBS), 2-Hydroxy-3-[tris(hydroxymethyl)methylamino]-1-propanesulfonic acid, N-[tris(hydroxymethyl)methyl]-3-amino-2-hydroxypropanesulfonic acid (TAPSO), Trizma, N-(2-Hydroxyethyl)piperazine-N-(2-hydroxypropanesulfonic acid) (HEPPSO), Piperazine-1,4-bis(2-hydroxy-3-propanesulfonic acid), dihydrate (POPSO), TEA, 4-(2-hydroxyethyl)piperazine-1-propanesulfonic acid (EPPS), Tricine, Gly-Gly, Bicine, N-(2-hydroxyethyl)piperazine-N′-(4-butanesulfonic acid) (HEPBS), 3-{[1,3-dihydroxy-2-(hydroxymethyl)propan-2-yl]amino}propane-1-sulfonic acid (TAPS), 2-amino-2-methyl-1,3-propanediol (AMPD), N-tris(hydroxymethyl)methyl-4-aminobutanesulfonic acid (TABS), N-(1,1-Dimethyl-2-hydroxyethyl)-3-amino-2-hydroxypropanesulfonic acid (AMPSO), N-cyclohexyl-2-aminoethanesulfonic acid (CHES), 3-(cyclohexylamino)-1-propanesulfonic acid (CAPSO), 2-methyl-2-amino-1-propanol (AMP), CAPS, and 4-(cyclohexylamino)-1-butanesulfonic acid (CABS). In some embodiments wherein the solvent contains Triton X, the concentration of Triton X may be between 0.001% and 0.1%. In some embodiments wherein the solvent contains BSA, the concentration of BSA may be between 0.05 and 0.5%.

The diameter of the second injector 450 may vary in size based on the sample type and sample volume. In some embodiments, the second injector 450 is a syringe, and the diameter of the syringe may range from 4 mm to 45 mm. In these embodiments, the maximum volume contained in the syringe may range from 0.5 mL to 200 mL. In some embodiments, both the injector 100 and the second injector 450 may both be syringes. In some embodiments, the second syringe may be the same size as the first. In some embodiments, the second injector 450 may be smaller than the first injector 100. In some embodiments, the size of the second injector 450 is not dependent on the size of the first injector 100.

In some embodiments, the solvent contained in the second injector 450 may be injected into the upper chamber 330 of the second filtration module 300 through the side channel connector 410. The microorganisms and/or microobjects captured on the second filtration membrane 322 are resuspended and homogenized in the solvent. In some embodiments, when the second injector 450 is a syringe, resuspension and homogenization may occur through sequential depression and pulling up of the syringe plunger. In other embodiments, resuspension and homogenization may occur through shaking of the two-module filter assembly 10. In yet other embodiments, resuspension and homogenization may occur through inversion of the two-module filter assembly 10. In other embodiments, resuspension and homogenization may occur through another mixing process. Following injection, the solvent with resuspended microorganisms and/or microobjects may be drawn up into the second injector 450 again. In some embodiments, the volume of solvent pulled back up may be the same as the volume ejected. In other embodiments, the volume of solvent pulled back up may be smaller than what was ejected. In some embodiments, the volume of solvent pulled back up may range from 25 to 300 μL. In some embodiments, the volume of solvent pulled back up may vary based on the membrane and methodology used.

### IX. Automated Processing Unit

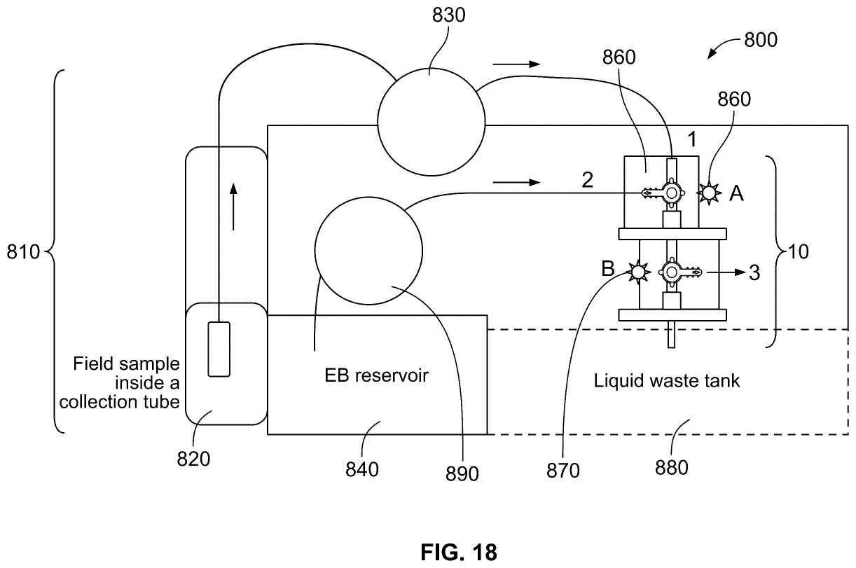

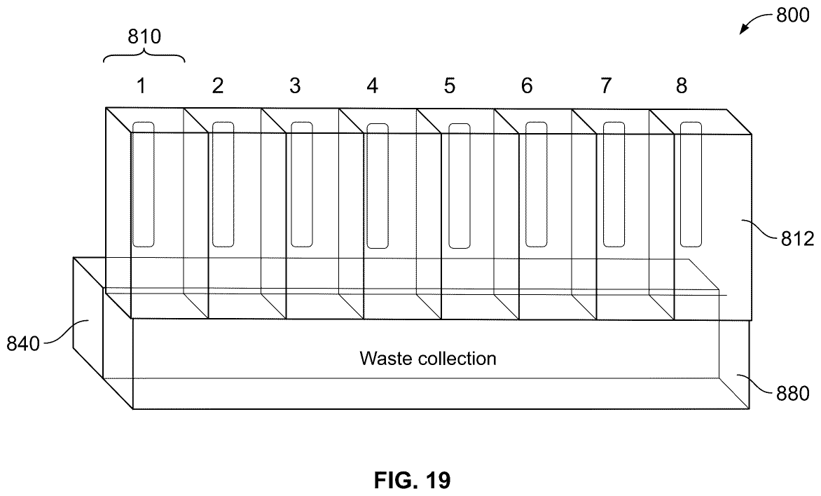

In another aspect, this invention relates to an automated processing unit 800, as depicted in FIGS. 18-19. An automated processing unit 800 comprises one or more processing modules 810, as well as a waste collection tank 880, a module frame 812, and an elution reservoir 840. The automated processing unit 800 provides automated, simple, and rapid processing of up to 20 samples at one time. Each processing module 810 comprises a collection tube 820, a sample pump 830, an elution pump 890, and a two-module filter assembly 10. In some embodiments, the sample pump 830 and the elution pump 890 are peristaltic pumps. In some embodiments, the sample pump 830 contains a liquid sample. In some embodiments, the sample pump 830 contains an air sample. The two-module filter assembly 10, as described above, comprises an injector 100, a first filtration module 200, a second filtration module 300, and a 3-way flow regulator 400. In some embodiments, the injector further comprises an input 3-way flow regulator 850 comprising an input 3-way valve 852, which can be changed in orientation to allow input from two different sources, the collection tube 820, and the elution reservoir, 840. The collection tube 820 is connected to the sample pump 830 by a fluid line. In some embodiments, the fluid line comprises silicon tubing. The sample pump 830 is further connected to the two-module filtration assembly 10 by a fluid line. In some embodiments, the fluid line comprises silicon tubing. In some embodiments, the fluid line from the sample pump 830 may be connected to the top input of the input 3-way flow regulator 850. The elution reservoir 840 is connected to the elution pump 890 by a fluid line. In some embodiments, the fluid line comprises silicon tubing. The elution pump 890 is further connected to the two-module filtration assembly 10 by a fluid line. In some embodiments, the fluid line from the elution pump 890 may be connected to the side input of the input 3-way flow regulator 850. In some embodiments, each processing module 810 further comprises a top motor 860 which controls the input 3-way valve 852, and a bottom motor 870, which controls the 3-way valve 440. In some embodiments the silicon tubing is connected to the two-module filter assembly 10 with Luer locks. In some embodiments, the input 3-way flow regulator 850 may comprise a combination of three Luer lock and/or Luer slip connections.

In some embodiments, the automated processing unit 800 is configured such that a plurality of processing modules 810 may operate at the same time, as shown in FIG. 19. A sample is pumped by the sample peristaltic pump 830 from the collection tube 820 through the two-module filter assembly 10, wherein microorganisms and/or microobjects are captured on the second filtration membrane 322. In some embodiments, between 3 and 20 mL of sample may be passed through the two-module filter assembly 10. Liquid or air is passed through the two-module filter assembly 10 and into the waste collection tank 880, which may be shared by a plurality of processing modules 810. Liquid from the elution reservoir 840, which may be shared by a plurality of processing modules 810, may be pumped through the two-module filtration assembly 10 by the elution peristaltic pump 890 after an amount of sample from the collection tuber 820 has been pumped through the two-module filtration assembly 10. In some embodiments, between 3 and 10 mL of liquid from the elution reservoir 840 may be passed through the two-module filter assembly. The top motor 860 controls the orientation of the input 3-way valve 852, which determines if liquid will be pumped by the sample peristaltic pump 830 from the collection tube 820 or by the elution peristaltic pump 890 from the elution reservoir 840. After liquid from the elution reservoir has been pumped into the two-module filtration assembly 10, the orientation of the 3-way valve 440 of the 3-way flow regulator 400 may be changed to provide access to the upper chamber 330 of the second filtration module 300 from the second filtration module upper connector 310. In some embodiments, the orientation of the 3-way valve 440 is changed by a bottom motor. The top and bottom motors

Following this, a second injector 450 may be attached to the second filtration module upper connector 310, and liquid from the elution reservoir 840 containing resuspended microorganisms and/or microobjects may be drawn into the second injector 450. In some embodiments, liquid may be drawn into the second injector 450 by reversing the direction of flow of the elution pump 890. In some embodiments, the second injector 450 may be a syringe or tube. In some embodiments, the volume drawn into the second injector 450 may range from 50 to 300 μL. In other embodiments, the volume drawn into the second injector 450 is the maximum volume held by the injector 450. In some embodiments, the sample to be processed in the automated processing unit 800 may be liquid, air, food, and swab samples. In some embodiments, one or more injector adapters 110 may be used in combination with the automated processing unit 800. In some embodiments, a liquid in the collection tube 820 may be inoculated with a swab or other field sample prior to processing.

The elution reservoir 840 may be filled with a buffer solution, which be selected from the group comprising a Tris buffer solution, a Tris-EDTA buffer solution, a Tris-EDTA-Tween buffer solution, a Tris-EDTA-Tween-Trehalose buffer solution, a Tris-EDTA-Tween-Trehalose buffer solution optionally comprising water, 0.1-100 mM Tris, 0.1-100 mM EDTA, 0.005-0.02% trehalose, and 0.002-2% tween, other buffer solutions of pH 7-9, and other buffer solutions. In some embodiments, the elution reservoir 840 may be filled with a buffer solution further with a swab, sponge, or otherwise solid sample suspended in the liquid sample.

### X. Sample Collection Bag

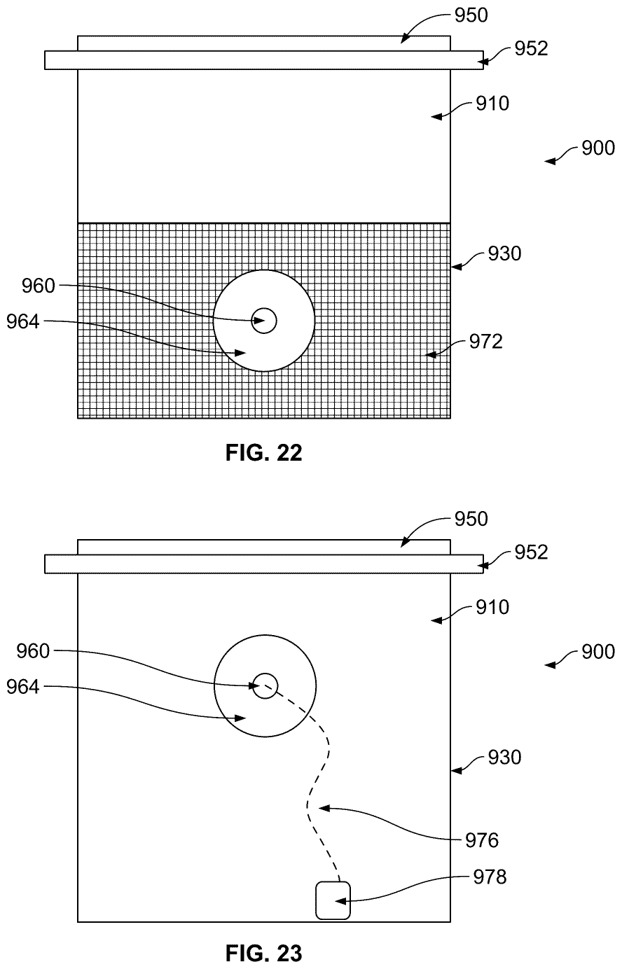

In another aspect, this invention relates to a sample collection bag 900, also referred to as a sampling bag 900, as described in FIGS. 22, 23, and 24. The sample collection bag 900 comprises front 910 and rear walls 920 connected at the side edges 930 and closed at the bottom, by any satisfactory manner. In other embodiments, the sample collection bag 900 may be formed from tubular stock. The walls are preferably formed of flexible material, such as plastic and polyethylene. The walls may have a thickness ranging from 0.1 to 5 mm. The front 910 and rear walls 920 define an internal cavity 940, with a top opening 950, extending between the side edges 930, providing access to the internal cavity 940.

The sample collection bag 900 may be employed in a variety of uses for containing an object or material, whether liquid or solid. A common application for the sample collection bag 900 is the collection of a field sample and preparation of a sample to be processed. In such applications, a sample may be added to the internal cavity 940 via the top opening 950, distending the bag. The sample collection bag 900 may be used in combination with the two-module filter assembly 10 to process samples to capture and concentrate microorganisms and/or microobjects.

The top opening 950 further comprises a deformable member 952, positioned against the outer surface of either the front wall 910 or the back wall 920, having two ends. The deformable member may be a round or flat wire or string, comprised of any combination of galvanized steel, stainless steel, iron, lead, and aluminum, or any other suitable material. The deformable member 952 may extend past the walls of the sample collection bag 900. An operator may use the deformable member 952 of the sample collection bag 900 to close and seal the internal cavity 940. One example of a method to close the bag comprises rolling the top of the top opening 950 down onto itself a plurality of times. The portion of the deformable member 952 extending past the walls may then be folded over against the rolled top opening 950 to close the top opening 950.

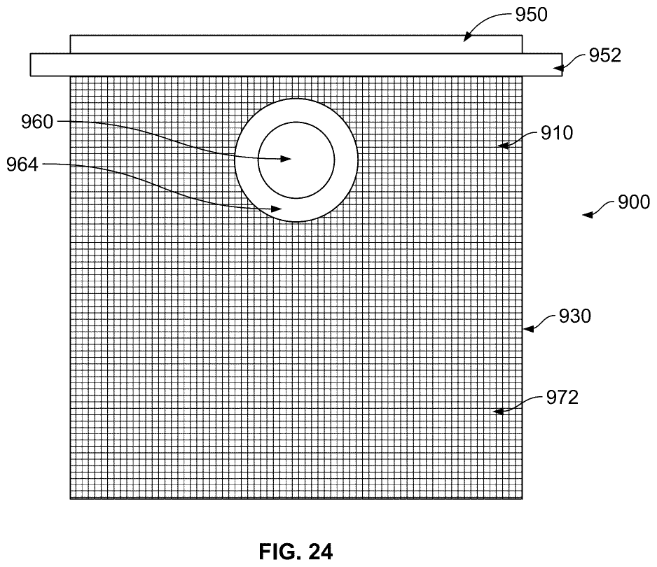

In some embodiments, the sample collection bag 900 further comprises an opening 960 positioned in the front wall 910. In some embodiments, the opening 960 further comprises a removable cap 962. In some embodiments, the opening 960 is located in the lower half of the front wall 910, as depicted in FIG. 22. In such embodiments, the opening 960 is in direct contact with the sample which is collected. In other embodiments, the opening 960 is located in the upper half of the front wall 910, as depicted in FIGS. 23 and 24. In such embodiments, the opening 960 is position such that it avoids direct contact with the sample which is collected.

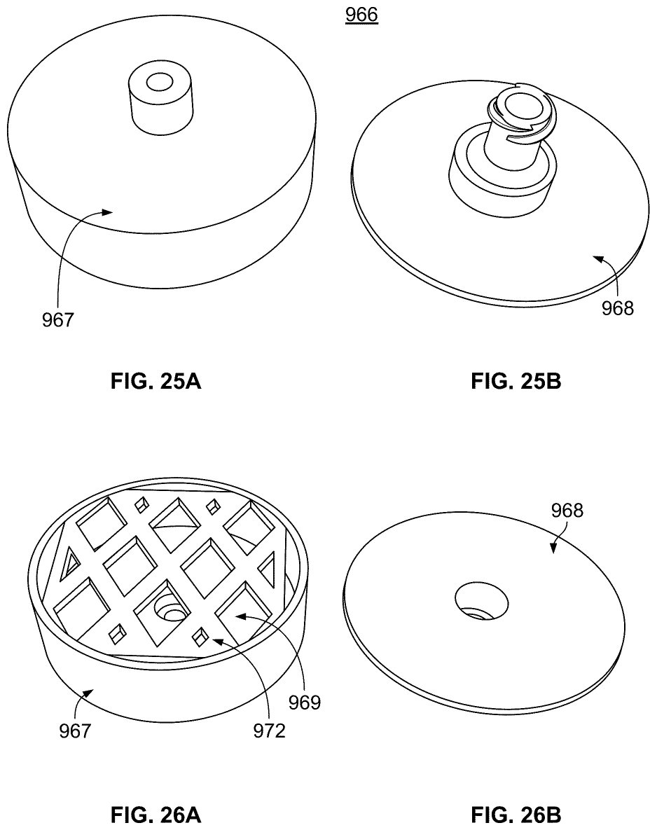

In some embodiments, the opening 960 comprises a plug 966. The plug 966 is made of an inside member 967 and an outside member 968, as shown in FIGS. 25A and 25B. The inside member 967 is situated in the internal cavity 940. The outside member 968 is situated outside of the bag. In some embodiments, the inside member 967 and outside member 968 are reversibly attached. In some embodiments, the two members are attached through a mechanism selected from the group comprising a slim-fit, snap-fit, or screwing mechanism, such that the front wall 910 of the collection bag 900 is disposed between and fitted snugly between the inside member 967 and outside member 968. In some embodiments, the diameter of the inside member 967 and outside member 968 are substantially the same. In some embodiments, the diameters of the inside member 967 and outside member 968 are about 20 mm to about 50 mm. In some embodiments, the diameters of the inside member 967 and outside member 968 are about 36 mm, as shown in FIGS. 25A, 25B, 26A, and 26B.

In some embodiments, the outside member 968 contains an opening to the outside of the sampling bag. In some embodiments, this opening is a Luer lock connector to attach a syringe or injector, as shown in FIG. 25B.

In some embodiments, the inside member 967 contains a cavity 969, as depicted in FIG. 26A. In some embodiments, the cavity is between about 5 and about 20 mm deep. In some embodiments, the cavity is about 10 mm deep. In some embodiments, prefilters are installed within the cavity 969 to avoid collecting large particles.

In some embodiments, the internal cavity 940 of the sample collection bag 900 is sterile. In such embodiments, a top seam 954 extends between the side edges 930, and a perforation 956 extends across the width of the front wall 910 and rear wall 920, between the top seam 954 and deformable member 952. In such embodiments, the portion of the sample collection bag 900 located above the perforation 956 is removed by an operator to form the top opening 950, the top opening 950 providing access to the sterile internal cavity 940 of the bag.

In some embodiments, the opening 960 comprises a female Luer lock with a removable cap, as depicted in FIGS. 22 and 23. In some embodiments, the opening 960 comprises a female Luer slip with a removable cap, as depicted in FIGS. 22 and 23. In other embodiments, the opening 960 comprises a screw cap closure with a removable cap, as depicted in FIG. 24. The opening 960 allows the operator to inject buffers and other liquids to suspend the collected sample. The opening 960 also allows the operator to collect an aliquot of the prepared sample for use with the two-module filter assembly 10. This mitigates the risk of cross-contamination arising from the otherwise necessity of reopening the top opening 950. It also limits the time necessary for sample processing. In embodiments in which the opening 960 comprises a female Luer lock or slip, a syringe may be inserted to simply and quickly collect an aliquot. This aliquot may then be transferred directly to the two-module filter assembly 10 described above.

In some embodiments, the area around the opening 960 is reinforced with an adhesive 964. The adhesive 964 is between 1 cm and 4 cm in diameter. The adhesive 964 reinforces the opening 960 to mitigate damages and leakage during manipulation of the bag.

In some embodiments, the sample collection bag 900 further comprises a filter 970 to remove large particles. In some embodiments, the filter 970 comprises a mesh sheet 972, as depicted in FIGS. 22 and 24. The mesh sheet 972 has a pore size between 40 and 300 μm. The mesh sheet 972 covers the opening 960. In some embodiments, the mesh sheet 972 is sealed into both side edges 930 and into the bottom seal. In some embodiments, the mesh sheet 972 extends substantially halfway up the bag. In other embodiments, the mesh sheet 972 extends substantially to the top opening 950. In some embodiments, the mesh sheet 972 is additionally sealed to the front wall 910 at the top opening 950 of the bag, on the same wall as the opening 960. In some embodiments, the mesh sheet 972 is a prefilter installed within the cavity 969 of the inside member 967, as depicted in FIG. 26A.

In other embodiments, the filter 970 comprises in in-line filter 974, as depicted in FIG. 23. The in-line filter 974 comprises a tube 976 which is connected to both the opening 960 at the internal face of the front wall 910 and a filter 978. The tube 976 is long enough such that it easily stretches to the bottom of the internal cavity 940. In some embodiments, the tube 976 is between 4 cm and 5 cm in length. In some embodiments, the filter 978 is any appropriate in-line filter and is selected from the list comprising a porous stone with pore size>100 μm, a membrane filter, a sponge filter, an in-line filter, and a mesh filter.

The sample collection bag 900 described above overcomes operation and practical challenges of other sample collection bags. For example, the opening 960 limits the necessity of opening the bag multiple times, allowing for faster, easier, and safer sample preparation and processing. Furthermore, the filter 970 overcomes technical and analytical drawbacks by preventing the collection of large particles that would otherwise clog the collection apparatus (pipette, syringe, otherwise injector). This provides greater accuracy and consistency in collection volume. The smaller size of the opening 960 compared to the top opening 950 minimizes the risk of contamination and spills. When compared to other traditional bags, the sample collection bag 900 prevents operator fatigue and minimizes the chance of error. The features of the sample collection bag 900 allows for a smaller bag to be used, minimizing waste and increasing compactness, impacting mobile and field-based testing workflows.

### XI. Methods for Concentrating Microorganisms and Microobjects

In another aspect, the invention relates to a method of concentrating microorganisms and microobjects from dilute samples comprising: a) loading a sample into an injector; b) connecting an injector to a two-module filter assembly, wherein the two-module filter assembly comprises a first filtration module, a second filtration module, and a 3-way flow regulator; c) injecting the sample into the two-module filter assembly; d) capturing larger particles within the first filtration module; e) capturing microorganisms and microobjects within the second filtration module, and; f) resuspending the microorganisms microobjects in a solvent.

In another aspect, the invention relates to a method of concentrating microorganisms and microobjects from dilute samples comprising: a) loading a sample into an injector, b) connecting the injector to the connector at the top of the first filtration module, c) depressing the plunger on the injector between one and three times to pass the sample to be analyzed through the first and second filtration modules, capturing the microorganisms and microobjects on the filter of the second filtration module, d) changing the orientation of the 3-way flow regulator to seal the bottom of the first filtration module and allow access to the upper chamber of the second filtration module, e) attaching a second injector containing a solvent, which may be selected from the group comprising one or more water, a buffer solution, a Tris(hydroxymethyl)aminomethane (Tris) buffer solution, ethylenediaminetetraacetic acid (EDTA), Tween, Trehalose, a Tris-EDTA-Tween-Trehalose buffer solution, Triton X, bovine serum albumin (BSA), other buffer solutions of pH 7-9, and other buffer solutions, to the side channel of the 3-way flow regulator and injecting the solvent into the upper chamber of the second filtration module, f) resuspend and homogenize the microorganisms and microobjects captured on the filter of the second filtration module, g) draw up the solvent with resuspended microorganisms and microobjects into the second injector, h) transferring the solvent with resuspended microorganisms and microobjects to a receptacle for further analysis.

In another aspect, the invention relates to a method of concentrating microorganisms and microobjects from dilute samples and detecting pathogens, comprising: a) loading a sample into an injector, b) connecting the injector with the connector at the top of the first filtration module, c) depressing the plunger on the first injector between one and three times to pass the sample to be analyzed through the first and second filtration modules, capturing the microorganisms and microobjects on the filter of the second filtration module, d) changing the orientation of the 3-way flow regulator to seal the bottom of the first filtration module and allow access to the upper chamber of the second filtration module, e) attaching a second injector containing a solvent, which may be selected from the group comprising one or more of water, a buffer solution, a Tris(hydroxymethyl)aminomethane (Tris) buffer solution, ethylenediaminetetraacetic acid (EDTA), Tween, Trehalose, a Tris-EDTA-Tween-Trehalose buffer solution, Triton X, bovine serum albumin (BSA), other buffer solutions of pH 7-9, and other buffer solutions, to the side channel of the 3-way flow regulator and injecting the solvent into the upper chamber of the second filtration module, f) resuspend and homogenize the microorganisms and microobjects captured on the filter of the second filtration module, g) draw up the solvent with resuspended microorganisms and microobjects into the second injector, h) transferring the solvent with resuspended microorganisms and microobjects to a PCE tube preloaded with isothermal reagents, i) incubating the contents of the PCE tube in a microanalyzer isothermal amplification fluorescence detector such as the ALADDIN ANALYZER™ for up to 60 minutes.

In another aspect, the invention relates to a method of concentrating microorganisms and microobjects from dilute samples, comprising: a) inserting the syringe adapter into an injector pre-filled with liquid, b) inserting an inoculated swab into the adapter and moving it up and down up to ten times, c) disposing of the swab and injector adapter, d) connecting the first syringe to the connector at the top of the first filtration module, c) depressing the plunger on the injector between one and three times to pass the sample to be analyzed through the first and second filtration modules, capturing the microorganisms and microobjects on the filter of the second filtration module, d) changing the orientation of the 3-way flow regulator to seal the bottom of the first filtration module and allow access to the upper chamber of the second filtration module, e) attaching a second injector containing a solvent, which may be selected from the group comprising one or more of water, a buffer solution, a Tris(hydroxymethyl)aminomethane (Tris) buffer solution, ethylenediaminetetraacetic acid (EDTA), Tween, Trehalose, a Tris-EDTA-Tween-Trehalose buffer solution, Triton X, bovine serum albumin (BSA), other buffer solutions of pH 7-9, and other buffer solutions, to the side channel of the 3-way flow regulator and injecting the solvent into the upper chamber of the second filtration module, f) resuspend and homogenize the microorganisms and microobjects captured on the filter of the second filtration module, g) draw up the solvent with resuspended microorganisms and microobjects into the second injector, h) transferring the solvent with resuspended microorganisms and microobjects to a receptacle for further analysis.

In another aspect, the invention relates to an automated method of concentrating microorganisms and microobjects from dilute samples, comprising: a) injecting a liquid sample into a two-module filter assembly, and; b) injecting an elution solvent into a two-module filter assembly. In some embodiments, the method may further comprise drawing up the solvent from the second filtration module.

In other embodiments, the invention relates to a kit for point-of-care system for the detection of pathogenic microorganisms and microobjects, including a sterile injector for unfiltered sample uptake, a sterile two-module filter assembly described above enclosed in a cylindrical shell, a sterile second injector for filtered sample uptake, a sterile PCR tube preloaded with isothermal reagents, an apparatus for testing DNA, and optionally a sterile injector adapter and swab for solid samples. In some embodiments, the apparatus for testing DNA is an ALADDIN ANALYZER™.

### XII. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Salmonella as used herein refers to a genus of rod-shaped, predominantly motile, enteromicroorganisms. It can be found in animal, human, and non-living habitats.

As used herein, “Serovar” or “Serotype” is the short form of referring to the serological variants of microorganisms, and is a way to distinguish between distinct types of microorganisms that fall within a single species. The particular serovar of a strain refers to the individual classification of that microorganisms within the species, as based upon cell membrane antigens.

Escherichia coli (E. coli) as used herein is a Gram-negative, rod-shaped, facultative anaerobic bacterium. Most E. coli strains harmlessly colonize the gastrointestinal tract of humans and animals as a normal flora. However, there are some strains that have evolved into pathogenic E. coli by acquiring virulence factors (e.g., toxin genes) through plasmids, transposons, bacteriophages, and/or pathogenicity islands. The designation “STEC” refers to Shiga Toxin-producing E. coli strains that cause a serious diarrheal disease in humans.

As used herein, “point-of-care”, “point-of-contact” or “POC” or “point of use” refers to a location at or near the location where the diagnostic system is used. A POC diagnostic system can be performed at the same place that the sample was collected. Examples as provided below are not intended to be limiting examples of locations where the POC diagnostic system may be used. In the case of human disease diagnostics, it refers to tests that do not require central laboratory facilities or highly trained technicians; and thus, can be done at home, in schools, at pharmacies, and many other locations. In the case of animal disease, it refers to tests that can be done on a farm, in a veterinarian's truck, in a veterinary clinic, in the owner's home, or any reasonable location close to the test animal. In the case of food safety, it refers to diagnostic tests that can be done in farm fields, in food storage facilities, in areas where food processing occurs, at abattoirs, at grocery stores, in restaurants, at import/export regulatory facilities, in homes, and many other places close to at-risk foods. In the case of plant pathology (fruits, vegetables, crops, forests and trees, ornamentals, gardens, golf courses, flowers, mushrooms, other plants), it refers to tests that can be done in the field or close to the field where plants are produced, in plant processing facilities, storage facilities, greenhouses, various transportation system for plants, grain and plant products as well as various places where plants are processed, stored, conditioned and shipped. In the case of water analysis, it refers to tests that can be done in places where analysis needs to be done on dormant or circulating water, cleaning water, run-offs, sewage, and any type of water system that can be contaminated with biological agents.

As used herein, “primer set” refers to short synthetic DNA oligonucleotides (<100 bp) used in LAMP reactions. The primer set is composed of 5-6 individual primers, and these are what amplify a specific region of the DNA or RNA target.

As used herein, “amplifying” and “amplification” refers to a broad range of techniques for increasing polynucleotide sequences, either linearly or exponentially. Amplification methods may be performed isothermally such as Loop-mediated isothermal amplification (LAMP). In various embodiments, the term “amplification product” or “amplified product” includes products from any number of cycles of amplification reactions.

As used herein, the terms “microobjects” and “microorganisms” refer to self-organized, physiologically integrated, and functionally autonomous biological systems, and includes but is not limited to fungi, parasites, bacteria, archaea, protists, and other single-celled microorganisms. The term also encompasses viruses as well as other contaminants and pathogens.

As used herein, “connector” refers to a mechanical connection interface configured to couple two fluid- or air-conveying components. In some embodiments, one component includes a male projection having a tapered surface and none or any number of external threads or ridges, and the mating component includes a female receptacle with a corresponding internal geometry to engage the projection, optionally via rotational or threaded locking mechanism. The “connector” provides a secure, fluid-tight seal upon engagement.

### XIII. Food Contamination

Infectious diseases in animals are costly by reducing animal productivity and requiring a number of treatments. As an example, it is estimated that parasitic worms cost the European livestock industry more than €1.8 billion per year, with drug-resistance costing at least €38 million per year in production losses and treatment costs. Biological contamination in the food chain is the cause of foodborne diseases. According the USFDA Foodborne diseases affect 48 million people in the USA every year, resulting in 128,000 hospitalizations and 3,000 deaths. It is estimated that 60% of food poisoning happens in restaurants. These diseases cost $55 billion a year to the US economy (Kowitt, B., Fortune Magazine (2015)). The pharmaceutical industry could also bear high costs due to biological contamination as these examples demonstrate: a contamination in Genzyme's manufacturing plant costs the company $300m in lost revenue in addition to $175m fine by the US Food and Drug Administration; after a major contamination, Johnson & Johnson had to refit its manufacturing plant costing more than $100m, in addition to the recall and reaction from the market that cost the company $1.6 bn.

Contamination of foodborne pathogens in the food production environment causes huge economic losses, attributed to waste of raw food materials, and poses a significant public health issue that can weaken the agricultural manufacturing sector. The presence of Enterobacteriaceae on food provides an indicator of the quality of a food and hygiene conditions of its processing. The Enterobacteriaceae family includes a number of important foodborne pathogens, including Salmonella, pathogenic Escherichia coli, Shigella spp., Yersinia enterocolitica, and others, which may cause both foodborne illness and food spoilage (Baylis, C., Uyttendaele, M., Joosten, H., Davies, A., & Heinz, H. J. (2011). The Enterobacteriaceae and their significance to the food industry. In ILSI Europe Report Series.) Livestock such as cattle, poultry, and swine are known reservoirs of Enterobacteriaceae such as E. coli. and Salmonella.

According to a risk assessment analysis done between Apr. 8, 2020-Feb. 8, 2022, 30% of ground pork products in retail settings were found to be contaminated with Salmonella, creating a huge risk of food borne illness to end consumers (USDA, Federal Register Notice Docket No. FSIS-2019-0023202 (2020)). In addition, there is a growing demand for testing genetically modified microorganisms present in the food or feed samples to meet the appropriate national and international controls, performing independent verification to trade in confidence with countries specifying GMO-free products, and preventing cross-contamination throughout the supply chain. The global GMO testing market is expected to grow from $1.85 billion in 2021 to $2.08 billion in 2022 at a compound annual growth rate (CAGR) of 12.3%. (GMO Testing Global Market Report October 2022).

### A. Quantifying Salmonella Contamination

Salmonella is a leading cause of foodborne illness, with 1.3 million cases of salmonellosis occurring annually in the U.S. (Bearson, S. M. D., Annu Rev Anim Biosci., 10:373-393 (2022)). Salmonella enterica is the type species and is further divided into six subspecies with S. enterica ssp. enterica as subspecies that includes over 2500 serovars. These serovars are ubiquitous in the environment and can colonize food producing animals and poultry as well as wild animals and birds without causing overt disease. Examples of Salmonella serovars include, but are not limited to, S. enterica serovar Typhimurium, S. enterica serovar Choleraesuis, S. enterica serovar Heidelberg, S. enterica serovar Paratyphi, S. enterica serovar Dublin, S. enterica serovar Derby, S. enterica serovar London, S. enterica serovar Enteritidis, S. enterica serovar Arizonae, S. enterica serovar Anatum, S. enterica serovar Berta, S. enterica serovar 4,[5],12:i:-, S. enterica serovar Agona, S. enterica serovar Braenderup, S. enterica serovar Infantis, S. enterica serovar Putten, S. enterica serovar Johannesburg, S. enterica serovar Eko, S. enterica serovar Schwarzengrund, S. enterica serovar Uganda, and S. enterica serovar Senftenberg.

Contamination of food products with Salmonella is not only a serious health issue, but also a significant economic impact to food producers with an annual cost of over $2 billion to the food industry (Magossi et al., 2019).

Contamination of food products with Salmonella is a recurring problem, causing at least 1 recall every year since 2010. According to a report from the FSIS for calendar year 2021, over a million pounds of food product was impacted from only 4 Salmonella-based food recalls. Pork products are especially susceptible to contamination, with over 30% comminuted pork and 9% of pork cuts confirmed to contain Salmonella (USDA Federal Register Notice Docket No. FSIS-2019-0023202 (2020)). Salmonella is found not only in finished food products but also at swine farms. A recent study from 2022 found that Salmonella was present in 11.3% of healthy pigs (Karabasanavar et al., Brazilian Journal of Microbiology, 53:1039-1049 (2022)). Simply segregating these infected pigs before harvest based on symptomatic visual cues is not possible, as these were all otherwise healthy and non-diarrheic. Instead, direct detection of Salmonella is required to determine if the bacterium is present in pigs before slaughter.

Salmonella is a major problem for poultry producers as well. Between 1998 and 2008, poultry accounted for 17.9% of foodborne illnesses in the United States, with Salmonella ser. Enteritidis and Typhimurium are responsible for 17.4% and 34% of poultry-related foodborne illnesses, respectively (Painter J. A., et al., Emerg. Infect. Dis. 2013; 19:407). An adequate diagnostic and disease prevention program is essential to a profitable commercial poultry operation.

Salmonella is shed in the feces of infected animals. Salmonella deposited in feces on soil can survive for long periods of time and can spread to adjacent areas through the blowing dust. The recent Salmonella contamination found in flour is believed to have been caused by wheat contamination by soil and dust from contaminated field (Magallanes López, A. M., & Simsek, S. (2021), Cereal Chemistry, 98(1), 17-30). Fecal contamination of ground water and drinking water can lead to Salmonella infection of people (Popa, G. L., & Papa, M. I. (2021), Germs, 11(1), 88). The recent outbreaks of Salmonella due to melons is likely due to contamination from soil or water that was contaminated by feces from Salmonella-infected animals.

B. Quantifying E. coli Contamination

The digestive tracts of humans and certain animals are known reservoirs of Escherichia coli. Enteropathogenic E. coli specifically has the potential to cause dangerous diarrheal illnesses. Verocytotoxin-producing E. coli (VTEC) describes strains of E. coli which produce Verocytotoxins or Shiga toxins. These toxins can damage the intestinal lining, leading to dangerous conditions such as hemorrhagic colitis, which can cause renal failure and hemolytic anemia. E. coli O157:H7 is commonly known to produce Shiga toxins and cause severe illness. The US FDA (Food and Drug Administration) has identified six additional serogroups, E. coli O26, O45, O103, O111, O121, and O145, as the most-commonly identified types outside of O157 causing foodborne illness (Bertoldi, B., Richardson, S., Goodrich Schneider, R., & Schneider, K. R. (n.d.). Preventing Foodborne Illness: E. coli “The Big Six”). However, most strains of E. coli such as E. coli Nissle 1917 are harmless. Thus, the development of new tests distinguishing pathogenic and non-pathogenic microorganisms, including E. coli is imperative to quantifying dangerous contamination (Lorenz, et al., Analytical and Bioanalytical Chemistry, 412:8241-8247 (2020)).

E. coli outbreaks also present a large economic concern. Cases of E. coli are estimated to cost approximately $306 million per year, mainly due to lost production from premature death (Rosson, P., & Adcock, F. (n.d.). Economic Impacts of E. coli on U.S. Beef.)

E. coli shedding from animals and wildlife is seasonal, having peaks in the summers. In addition to animals themselves, feces, insects, birds, water, animal feeders, and other environmental sources can act as reservoirs of E. coli (McClure, World Journal of Microbiology & Biotechnology 16:749-755 (2000)). One important control measure for E. coli and especially O157, is heating. However, this is difficult to control over a large subject group, and some products, such as fermented meat, is not meant to be heated before consumption. Thus, testing for E. coli and disinfection or other measures of E. coli removal are paramount to human health.

### C. Pork Manufacturing and Lairage Pen Contamination

The pork manufacturing process begins with a shipment of pigs to the meat packing plant where they are held in lairage, a pre-harvest transient holding pen. These transient pre-harvest lairage pens is one area where Salmonella is spread amongst other members of the herd immediately prior to the food manufacturing process (Vieira-Pinto et al., International Journal of Food Microbiology, 110(1):77-84 (2006)). Along with potential amplification in lairage, subclinical pigs are harvested and contaminated trim meat is combined from multiple sources. This trim is then ground, potentially contaminating the meat from Salmonella-negative pigs.

Plants are required to test 5 times a month, and the focus of these diagnostics is on post-harvest final products. The current testing paradigm for pork manufacturers is proprietary and contained in their HACCP (Hazard Analysis and Critical Control Point) plans. But an example procedure, as described by the FSIS, includes a preliminary identification test followed by a culture-based selection for Salmonella and MALDI-based serotype identification (USDA, MLG 4 Appendix 2.06 (2021)). Overall, the process can take anywhere from about 16 hours to 6 days. For preliminary identification tests, the majority of the testing duration is due to a pre-enrichment culture step, which can take 15 hours or longer. Use of these tests for pre-harvest detection would be ineffective at responding to the short lairage holding durations, which can be as quick as 1 hour. The processing of beef also uses a lairage system, where the spread of both Salmonella and E. coli contamination is of concern.

### D. Beef Manufacturing

Over 13 years from 1990 to 2003, 438 foodborne illness outbreaks were linked to beef and beef-containing products, 43% of which involving Escherichia coli and Salmonella (Algino et al., Journal of Food Science, 72(5): M173-M179 (2007)). Beef is a common source of Salmonella illness. From 2012-2019, beef accounted for up to 9% of all Salmonella illnesses (Canning, et al., Journal of Food Production, 86(5):100071 (2023)).

The USDA FSIS (Food Safety and Inspection Service) has mandated a number of microbial tests based on the estimated daily volume of raw beef per day (USDA FSIS Directive 10,010,1 Rev. 6 (2024)). In the case of beef carcasses, testing for indicator microorganisms such as E. coli and Salmonella is conducted daily (Amsa. (n.d.). The Role of Microbiological Testing in Raw Beef Food Safety Programs The Scientific Perspective). Studies can often be long-term, depending on their results, with samples being required at different points, such as beef carcasses, subprimals, beef trimmings, and raw ground beef, which can be both labor and cost prohibitive.

### E. Poultry Manufacturing

The consumption of poultry meat worldwide is increasing, especially in the USA, with chicken being the most consumed. Thus, there is an increasing need for the health and safety of poultry meat, especially chicken. Pathogenic microorganisms can be introduced to carcasses, cuts, and processed meat products at many locations along the production process, especially from surfaces, air, and liquids in slaughterhouses. From 1998-2012, poultry was the leading cause of foodborne outbreaks in the USA (Rouger, et al., Micromicroorganisms, 5(3):50 (2017)).

Regulations from the USDA have imposed minimum requirements for the frequency and location of poultry sampling. These regulations are based on the size of the organization and the volume of meat produced. For all organization sizes, the minimum frequency of sampling is once during each week of operation, with a sample selected at post-chill, meaning after the poultry carcasses exit the chiller after slaughter but before further processing. Most organizations are also required to sample once a week pre-chill. The frequency of these tests, and the length of their testing, imposes a burden on manufacturing organizations. Furthermore, often, samples must be transported off-site to a separate laboratory, delaying the results of these tests by up to 24 hours. This length of transport may also impose questions about accuracy of these tests (“FSIS Compliance Guideline: Modernization of Poultry Slaughter Inspection. Microbiological Sampling of Raw Poultry” (2015) https://www.fsis.usda.gov/sites/default/files/import/Microbiological-Testing-Raw-Poultry.pdf). A rapid, on-site, easy test for pathogenic microorganisms would provide a great advance in testing for these manufacturing organizations.

### XIV. LAMP Technology

Loop-mediated isothermal amplification (LAMP) is a nucleic acid-based technology that can selectively amplify a target DNA sequence using a set of up to six primers, recognizing six to eight regions of the target DNA sequence-hence a high specificity, and strand displacement polymerase under isothermal conditions. The auto-cycling reactions lead to accumulation of a large amount of the target DNA and other reaction by-products, such as magnesium pyrophosphate, that allow rapid detection using varied formats (Njiru, PLOS Neglected tropical Diseases, 6(6):e1572 (2012)).

LAMP is well known for its robust and highly sensitive and specific amplification of target DNA, which is achieved by utilizing the set of five to six primers. Moreover, LAMP excels through its isothermal and energy efficient amplification requirements, rendering it a prime candidate for low-cost diagnostics and analysis at the point of need. This technology fits with the recommendation of the WHO for a molecular test suitable for developing countries, and by extension for wider and more frequent usage in developed countries. The World Health Organization (WHO) recommends that an ideal diagnostic test suitable for developing countries should be Affordable, Sensitive, Specific, User-friendly (simple to perform in a few steps with minimal training), Robust and rapid (results available in less than 60 min), Equipment free, and Deliverable to the end user (ASSURED).

Following these guidelines, it is clear that this technology has many benefits over the current technologies to address the unmet needs as described above. The technology is affordable and does not require expensive thermal cycling devices that are necessary for qPCR. The nucleic acid amplification can be done at 60-70° C., and optimally at 65° C., using simple heating devices that do not require skilled operators. The technology is also sensitive, having the same sensitivity as qPCR by using the series of 4-6 primers which increase the sensitivity to the method. It is also specific, having the same specificity as qPCR—it amplifies specific genes that are unique to the pathogen of interest. The technology also provides the advantage of the ability to developed new primers quickly. For pathogens with high levels of genetic variability, the LAMP test can be quickly modified to detect new strains. LAMP is also user-friendly, not needing complex equipment, having a reduced number of steps to prepare and process the samples, allowing simple reading of the results (positive results can be visualized by a color change, fluorescence generated after intercalation of a dye into DNA, or the presence of turbidity (cloudiness) that can be visualized with the naked eye). The technology is robust and is rather forgiving for sample purity because LAMP typically uses Bst 3.0 polymerase, which is capable of polymerizing DNA strands in the presence of inhibitors. It is therefore well suited to perform in “dirty” environments: at the farm; in processing plants; on the manufacturing floor. Results are rapidly obtained in under one hour. The technology has the advantage of requiring low-cost equipment, with no need for a complex thermocycler. The only equipment is a combination of a heating block and a reader, which can be combined in a small and compact “box’. The technology provides to the end user the small equipment (“box”) which is portable, light and rugged to be deliverable at POC facilities (farms, manufacturing plants, etc.).

### XV. Concentration of Microorganisms from Dilute Samples Using a Syringe Filter

The food industry relies on extremely sensitive pathogen detection technologies to ensure that food products are safe for consumers. Sensitive detection of pathogens is especially important for environmental samples taken from the surfaces surrounding food production areas, since these samples may contain less than 10 microorganisms in a 10 cm×10 cm sampling area. Due to the rapid multiplication of microorganisms every 20 minutes to two hours, even a small number of microorganisms can quickly surpass a minimal infectious dose. Current diagnostic methods use this rapid growth to enrich microorganism populations in food samples in a method known as “bacterial culture” (or just simply “culture”). Increasing the total number of microorganisms in a sample greatly simplifies downstream detection assays. The biggest issue to using culture methods is the time involved. Typically, these cultures are grown over 18 hours to maximize the number of microorganisms from a sample. Release of food products or manufacturing facilities may depend on results from these time-consuming diagnostic processes. Long sample processing times, like overnight culture methods, reduce the shelf-life of food products, which may be as low as 3 to 5 days for some fresh meat products (A. K. Magoulas & CiCi Williamson, USDA's Food Safety and Inspection Service in Health and Safety, Aug. 19, 2014).

Disclosed herein is a novel microorganismal concentration method that can be performed in less than 5 minutes, thereby avoiding long sample processing times for microorganismal targets. The method uses a two-module filtering assembly to concentrate microorganisms from dilute samples. In some embodiments, the method comprises loading a sample into a syringe connected to the two-module filtering assembly. Depressing the plunger forces the liquid through the syringe. In the first filtration module of the assembly, particles>10 μm are filtered off. With pressure from plunger depression, the sample flows out of the first module into a 3-way flow regulator, which directs the sample into the second filtration module of the assembly. In the second filtration module of the assembly, the sample flows through a second filter, while microorganisms are captured on the surface. A 0.1-0.8 μm membrane filter is known to be appropriate for capturing microorganisms during filtration (Millipore Sigma, “Effect of Membrane Filter Pore Size on Microbial Recovery and Colony Morphology,” Technical Bulletin). The pore size of the membrane filter may be altered based upon the type of microorganisms desired for capture. After the entire sample has passed through the two-module filtering assembly, the orientation of the 3-way is changed to seal the bottom of the first filtration module and open the second filtration module to a side channel of the 3-way, comprising a connector. A second syringe containing a solvent is attached to this side channel, and its plunger is depressed to pass the solvent into the upper chamber of the second filter membrane. The plunger of the second syringe is moved back and forth several times and up to 10 times to resuspend and homogenize the microorganisms. Then, this resuspended mixture is pulled back into the plunger and transferred for further analysis. This further analysis may comprise analysis with a microanalyzer isothermal amplification fluorescence detector such as the ALADDIN ANALYZER™

The disclosed method poses many advantages compared to current microorganismal culture methods, such as: (1) completion of the sample preparation in less than 5 minutes compared to the up to 60 minutes needed for traditional sample preparation; and completion of detection in less than 60 minutes, compared to the 24-72 hours for traditional detection methods, (2) separation of the target microorganisms and nucleic acids from unwanted sample matrix components, (3) potential to capture any target larger than the syringe filter pore size, like microorganisms, parasites, viruses, fungi, and allergens, which cannot always be easily cultured, and (4) simple and easy access to concentrated microorganisms.

### EXAMPLES

### Example 1: Head-to-Head Performance Evaluation of the Hypercell Test for Core Enterobacteriaceae Versus the AOAC Microbiology Method on Artificially Inoculated Beef Carcass Swab

A precise Enterobacteriaceae (EB) test (Hypercell EB Core EZ) was developed that identifies only the most common and dangerous microorganisms—mainly Salmonella and E. coli—to help food processors focus on true health risks and avoid unnecessary action on less significant Enterobacteriaceae.

### Methodology

Beef primals (n=38) were inoculated with a known concentration of microorganisms (e.g., 10-10,000 CFU per mL) and homogenized for 30 seconds. Primals were then swabbed and processed to detect specific EB according to Hypercell's and the reference methods. Primals were also swabbed (n=6) prior to inoculation to determine the presence and load of pre-existing microorganisms in the samples, to avoid interference with the results. All analyses were conducted using a paired study design. The validation was conducted by three operators to confirm the reproducibility of the method.

Detection of EB in swabs using reference method (Association of Official Analytical Collaboration (AOAC)-certified 3M EB Petrifilm); One milliliter of each sample was plated in agar and inoculated plates were incubated at 35° C. for 24 hours before counting the colonies.

Detection of specific EB in swabs using Hypercell EB Core EZ method: 3 mL of each sample was processed using the two-module filter assembly described in the present application (<5 minutes). The concentrated purified product (50 μl) was transferred into isothermal reaction tubes and incubated in an Aladdin Analyzer™ fluorescent reader for 1 hour.

### Results

Similar detection accuracy was observed with Hypercell EB Core EZ tests compared to the AOAC method, but Hypercell EB Core EZ was faster: detection in less than one hour versus 24 hours (Table 1). All samples (n=32) spiked with a concentration of between 11 and 104 CFU/mL were detected positive using both the Hypercell (HCT: Hypercell EB Core EZ providing results in 60 minutes) and the AOAC microbiology (results after 24 hours incubation) tests.

No false results were detected using Hypercell tests (Table 1). EB was not detected in the blank samples (n=6) using both the Hypercell and AOAC microbiology tests.

TABLE 1
Detection of specific Enterobacteriaceae in carcass swabs across users.

| | Enterobacteriaceae prevalence | | | | | | | |
|---|---|---|---|---|---|---|---|---|
| Spiked | User 1 | | User 2 | | User 3 | | In this study | |
| concentration | (n = 11) | | (n = 15) | | (n = 12) | | (n = 38) | |
| (CFU/ml) | AOAC | HCT | AOAC | HCT | AOAC | HCT | AOAC | HCT |
| 0 | 0% | 0% | 0% | 0% | — | — | 0% | 0% |
| | (0/1) | (0/1) | (0/5) | (0/5) | | | (0/6) | (0/6) |
| 10-18 | 100% | 100% | 100% | 100% | 100% | 100% | 100% | 100% |
| | (1/1) | (1/1) | (1/1) | (1/1) | (3/3) | (3/3) | (5/5) | (5/5) |
| 85-103 | 100% | 100% | 100% | 100% | 100% | 100% | 100% | 100% |
| | (3/3) | (3/3) | (3/3) | (3/3) | (3/3) | (3/3) | (9/9) | (9/9) |
| 1.0k-1.2k | 100% | 100% | 100% | 100% | 100% | 100% | 100% | 100% |
| | (3/3) | (3/3) | (3/3) | (3/3) | (3/3) | (3/3) | (9/9) | (9/9) |
| 10k-12k | 100% | 100% | 100% | 100% | 100% | 100% | 100% | 100% |
| | (3/3) | (3/3) | (3/3) | (3/3) | (3/3) | (3/3) | (9/9) | (9/9) |

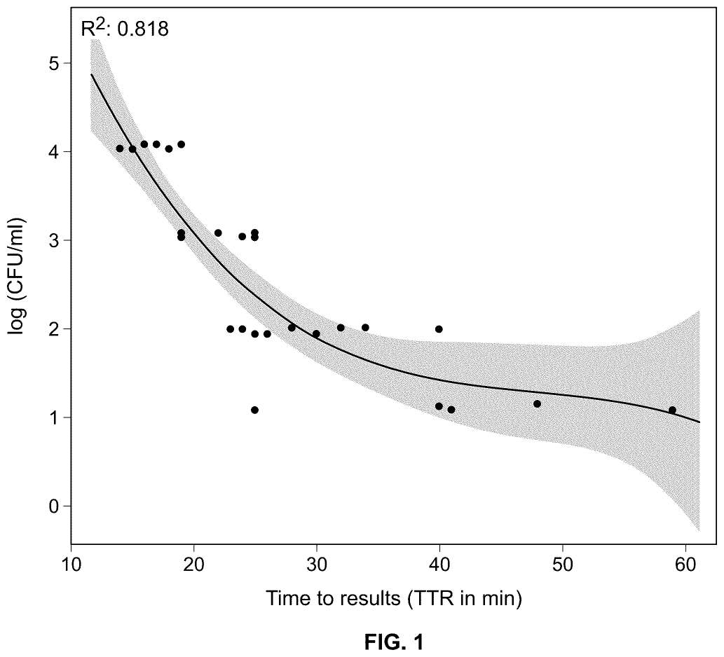

Hypercell EB Core EZ predicted with high accuracy the EB concentration in the field samples (FIG. 1). A strong correlation (average r2=0.82; FIG. 1) was observed between the Hypercell (detection of the fluorescent signal) and the AOAC microbiology method (CFU counts data). These results demonstrate that the Hypercell tests are a semi-quantitative method with equivalency to the AOAC method tested. As an example, in this study: the time to results of 20-25 minutes corresponded to a level of contamination of 1,000 CFU/mL.

FIG. 1 depicts that high quantification correlation was obtained between the AOAC EB microbiology method and Hypercell EB Core EZ data (r²=0.82). A sample was detected positive for EB using Hypercell when the fluorescent signal is recorded at a given time (time to results [TTR]).

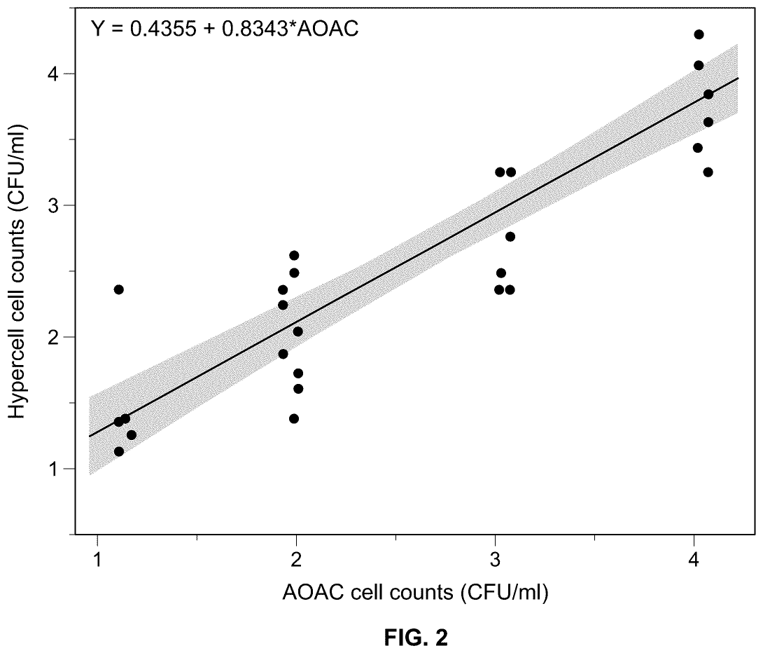

Hypercell EB Core EZ provided robust semi-quantitative data (FIG. 2). Results obtained with the Hypercell EB Core EZ were within 0.85-log±0.3 agreement with the counts obtained with the AOAC EB microbiology method, across the four concentrations tested.

FIG. 2 depicts predicted CFU counts obtained with the Hypercell EB Core EZ versus confirmed counts obtained with the AOAC EB microbiology method. Hypercell EB Core EZ counts were generated based on the linear correlation described in FIG. 1. AOAC EB microbiology counts were recorded based on plating data.

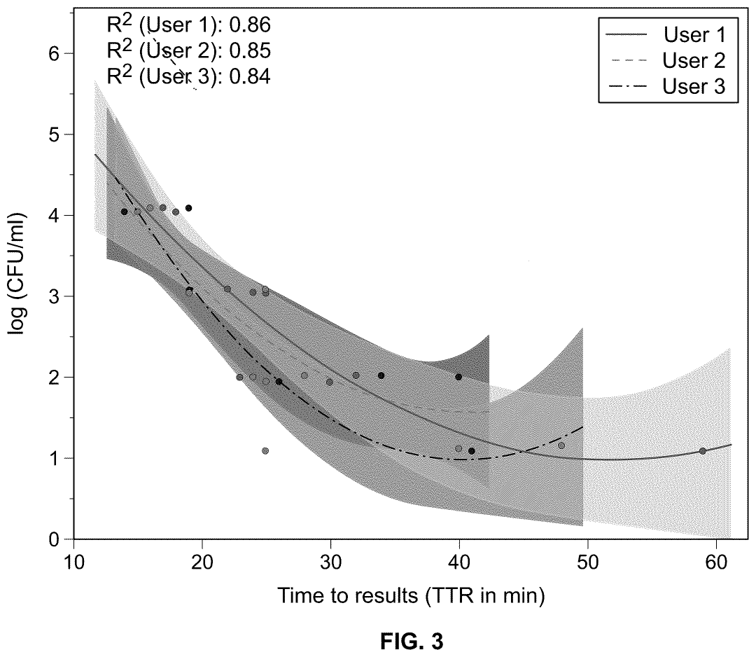

Very robust data was obtained across the three operators using the Hypercell EB Core EZ (FIG. 3). As demonstrated in FIG. 3, the three operators detected specific concentrations of EB in the field samples with high accuracy (user 1 r²=0.86; user 2 r²=0.85; user 3 r²=0.84). The disclosed tests are simple, rapid and provide robust results.

FIG. 3 depicts that Hypercell EB Core EZ provided robust results across the three operators. A sample was detected positive for EB using Hypercell when the fluorescent signal is recorded at a given time (time to results [TTR]).

### Example 2: Head-to-Head Performance Evaluation of the Hypercell EB Test for Enterobacteriaceae Versus the AOAC Microbiology Method on Artificially Inoculated Beef Carcass Swab

Hypercell EB test is similar to the current method used by food processors to determine the overall level of contamination without triggering a potential intervention from FDA or FSIS. But its scope of microorganisms identification is wider than the Hypercell EB Core EZ test that is only focused on the most dangerous Enterobacteriaceae.

### Methodology

Beef carcass swabs (the method of how meatpackers would sample the beef carcasses was simulated by purchasing pieces of beef and sampling them with a sponge; n=23) were inoculated with a known concentration of microorganisms (e.g., 8-14,000 CFU per mL) and homogenized for 30 seconds. Inoculated swabs were processed to detect specific EB according to the Hypercell EB and reference methods. Blank samples (n=5; supernatant before spiking with the microorganisms) were collected to assess the presence of pre-existing microorganisms in the samples, to avoid interference with the results obtained after spiking with a determined concentration of microorganisms. All analyses were conducted using a paired design. A total of 18 artificially spiked samples and five blanks were processed (n=4-5 samples per concentration).

Detection of EB in swabs using reference method (AOAC-certified 3M EB Petrifilm): One milliliter of field sample was plated in agar and inoculated plates were incubated at 35° C. for 24 hours before counting the colonies.

Detection of specific EB in swabs using Hypercell method: three milliliters of field sample were processed using the two-module filter assembly described in the present application (<5 minutes). The concentrated purified product (50 μl) was transferred into isothermal reaction tubes and incubated in the Aladdin Analyzer™ fluorescent reader for one hour.

### Results

Similar detection accuracy was observed with the Hypercell EB compared to the AOAC method, but Hypercell EB was faster, under 30 minutes versus the 24 hours for the AOAC method (Table 2). All samples (n=18) spiked with a concentration of between 8 and 10⁴ CFU/mL were detected positive using both the Hypercell EB (results under 30 minutes) and the AOAC microbiology (results after 24 hrs. incubation) tests.

No false results were detected using Hypercell EB (Table 2). EB was not detected in the blank samples (n=5) using both the Hypercell EB and the AOAC microbiology tests.

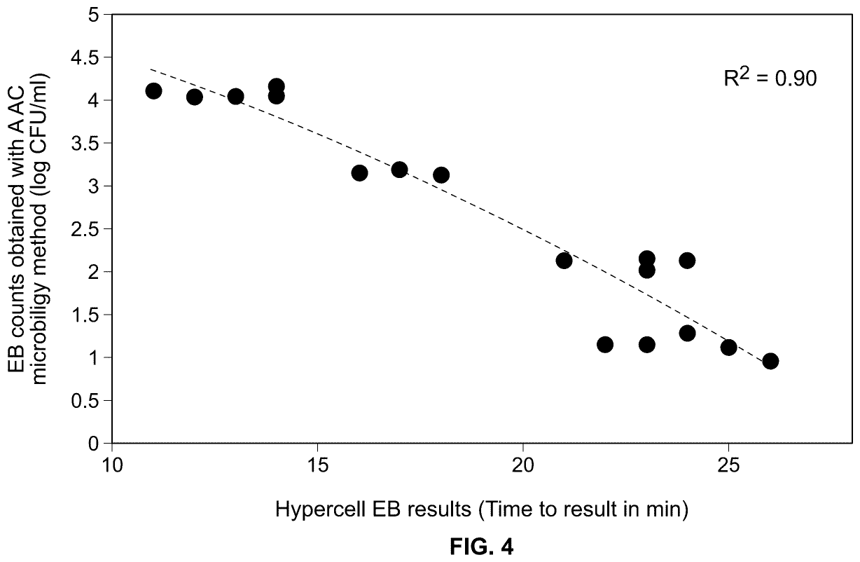

Hypercell EB predicted with high accuracy the EB concentration in the field samples (FIG. 4). A strong correlation (r²=0.90) was observed between the Hypercell (detection of the fluorescent signal) and the AOAC microbiology method (CFU counts data). Thereby, these results demonstrated that the Hypercell test is a semi-quantitative method with equivalent accuracy to AOAC microbiology method. As an example, for this study: a TTR of 16-18 minutes corresponds to a level of contamination of 1,000 CFU/ml.

TABLE 2
Detection of Enterobacteriaceae in carcass swabs.

| Spiked | Sample | | | | |
|---|---|---|---|---|---|
| concen- | size | Enterobacteriaceae prevalence | | | |
| tration | (total = | Microbiology (AOAC) data | | Hypercell data after | |
| (CFU/ml) | 23) | after 24 hrs. incubation | | 30 min reading | |
| 0 | 5 | 0% | (0/5) | 0% | (0/5) |
| 8-18 | 5 | 100% | (5/5) | 100% | (5/5) |
| 103-141 | 4 | 100% | (4/4) | 100% | (4/4) |
| 1.3K-1.5K | 4 | 100% | (4/4) | 100% | (4/4) |
| 11K-14K | 5 | 100% | (5/5) | 100% | (5/5) |

FIG. 4 depicts that high quantification correlation was obtained between the AOAC EB microbiology method and the Hypercell EB test (r²=0.90). A sample was detected positive for EB using Hypercell when the fluorescent signal is recorded at a given time (TTR).

### Example 3: Detection of Salmonella in Artificially Spiked Poultry Meat

### Methodology

Chicken samples (25 g of meat; n=37) were resuspended into a buffer (225 mL), inoculated with a known concentration of Salmonella (e.g., 10-220 CFU per g) and homogenized for 30 seconds. Inoculated samples were processed to detect Salmonella according to the Hypercell's and reference methods. Blank samples (n=10; supernatant before spiking with the microorganisms) were collected to assess the presence of pre-existing Salmonella in the samples, to avoid interference with the results obtained after spiking with a determined concentration of Salmonella. All analyses were conducted using a paired design. A total of 37 samples were processed for this study (4-5 samples per spiking dose; two independent experiments).

Quantification of Salmonella spiking dose used in chicken meat samples via microbiology plating method (XLT-4 agar): The same volume of inocula used for the spiking of the field samples was plated on agar and inoculated plates were incubated at 35° C. for 24 hours before counting the colonies. One milliliter of spiked field samples was also plated on agar as reference to assessing whether standard microbiology can detect low concentration of Salmonella in large samples. Similar incubation time and method described above were used.

Detection of Salmonella in chicken meat samples using Hypercell method: 10 milliliters of field sample were processed using the two-module filter assembly described in the present application (<5 minutes). The concentrated purified product (50 μl) was transferred into our isothermal reaction tubes and incubated in the Aladdin Analyzer™ fluorescent reader for one hour.

### Results

Hypercell Salmonella tests detected 100% of samples spiked with as little as 10 CFU/g of Salmonella in under 50 minutes without enrichment (Table 3) while the standard method only detected 67% of positive samples after 24 hours of incubation. All samples (n=27) spiked with a concentration of 10 CFU/g and above were detected positive using the Hypercell Salmonella test. On the other hand, only 18 out of 27 samples were detected positive for Salmonella using the microbiology method. The difference in LOD between the two methods is due to the higher input size (10 mL of field sample for Hypercell versus one milliliter of field sample for the microbiology method), which increases the probability of capturing a low number of microorganism cells in a larger volume of sample.

Hypercell Salmonella test provides robust and repeatable results over time (Table 3). Similar Salmonella prevalence data was obtained between two independent experiments conducted at different times.

No false results were detected using Hypercell Salmonella test (Table 3). Salmonella was not detected in the blank samples (n=10) using both the Hypercell and AOAC microbiology tests.

TABLE 3
Detection of Salmonella in poultry meat.

| Original | | Salmonella prevalence in poultry meat | | | | | | | |
|---|---|---|---|---|---|---|---|---|---|
| spiking | Sample | Microbiology data after | | | | Hypercell Salmonella test | | | |
| concentration | size | 24 hrs. incubation | | | | data after 60 min reading | | | |
| (CFU/g)* | (total = 37) | Rep 1 | | Rep 2 | | Rep 1 | | Rep 2 | |
| 0 | 10 | 0% | (0/5) | 0% | (0/5) | 0% | (0/5) | 0% | (0/5) |
| 10-15 | 10 | 40% | (2/5) | 60% | (3/5) | 100% | (5/5) | 100% | (5/5) |
| 35-60 | 9 | 60% | (3/4) | 80% | (4/5) | 100% | (4/4) | 100% | (5/5) |
| 100+ | 8 | 100% | (4/4) | 100% | (4/4) | 100% | (4/4) | 100% | (4/4) |

*10 CFU/g of meat = 1 CFU/mL of supernatant given the meat is diluted 1/10 in a buffer.
Prevalence: Colonies observed on agar plates or fluorescent signal detected using Hypercell test.

### Example 4: Detection of Salmonella in Artificially Spiked Beef Carcass Swabs

### Methodology

Beef carcass swabs (the method of how meatpackers would sample the beef carcasses was simulated by purchasing pieces of beef and sampling them with a sponge; n=28) were inoculated with a known concentration of microorganisms (e.g., 3-1,600 CFU per mL) and homogenized for 30 seconds. Inoculated swabs were processed to detect Salmonella according to Hypercell's recommendations. Blank samples (n=12; supernatant before spiking with the microorganisms) were collected to assess the presence of pre-existing microorganisms in the samples, to avoid interference with the results obtained after spiking with a determined concentration of microorganisms. A total of 28 samples were processed (n=4 samples per concentration; four independent experiments).

Quantification of Salmonella spiking dose used in samples via microbiology plating method (XLT-4 agar): The same volume of inocula used for the spiking of the samples was plated on agar and inoculated plates were incubated at 35° C. for 24 hrs. before counting the colonies. This method was used to verify the level of detection obtained using the two-module filter assembly described in the present application

Detection of Salmonella in samples using the Hypercell method: 10 mL of field sample was processed using the two-module filter assembly described in the present application (<5 minutes). The concentrated purified product (50 μl) was transferred into isothermal reaction tubes and incubated in the Aladdin Analyzer™ fluorescent reader for 1 hour. Results obtained with the Hypercell test (TTR in minutes) were compared to the bacterial counts obtained by direct plating of the inocula used for the spiking for the samples.

### Results

Hypercell Salmonella test detected 100% of samples spiked with as low as 3 CFU/mL of Salmonella under 60 minutes (Table 4). All samples (n=16) spiked with a concentration of 3 CFU/mL and above were detected positive using Hypercell Salmonella.

Hypercell Salmonella test provides robust and repeatable results (Table 4). Similar Salmonella prevalence and quantitative data were obtained across the four experimental repeats.

No false results were detected using Hypercell Salmonella test (Table 4). Salmonella was not detected in the blank samples (n=12) using both the Hypercell and the microbiology tests.

TABLE 4
Detection of Salmonella in beef carcass swabs.

| Original spiking | | | | | | | | | |
|---|---|---|---|---|---|---|---|---|---|
| concentration | Sample size | Hypercell Salmonella test data after 60 min reading | | | | | | | |
| (CFU/ml) | (total = 28) | Rep 1 | | Rep 2 | | Rep 3 | | Rep 4 | |
| 0 | 12 | 0% | (0/3) | 0% | (0/3) | 0% | (0/3) | 0% | (0/3) |
| 3-7 | 4 | 100% | (1/1) | 100% | (1/1) | 100% | (1/1) | 100% | (1/1) |
| 12-63 | 4 | 100% | (1/1) | 100% | (1/1) | 100% | (1/1) | 100% | (1/1) |
| 98-500 | 4 | 100% | (1/1) | 100% | (1/1) | 100% | (1/1) | 100% | (1/1) |
| 700-1,600 | 4 | 100% | (1/1) | 100% | (1/1) | 100% | (1/1) | 100% | (1/1) |

* Prevalence: fluorescent signal detected using the Hypercell test.

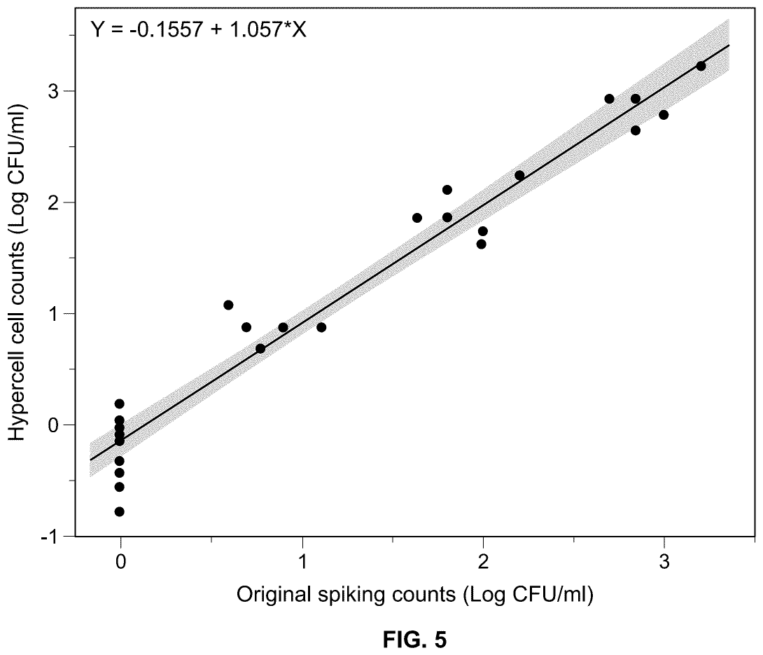

Hypercell Salmonella test predicted with high accuracy the concentration of Salmonella in the field samples (FIG. 5). A strong correlation (r²=0.93) was observed between the Hypercell kit (detection of the fluorescent signal) and the microbiology data (original spiking CFU counts).

FIG. 5 depicts that high quantification correlation was obtained between spiking dose and Hyeprcell Salmonella test (r²=0.93). A sample was detected positive for Salmonella using Hypercell when the fluorescent signal was recorded at a given time (TTR).

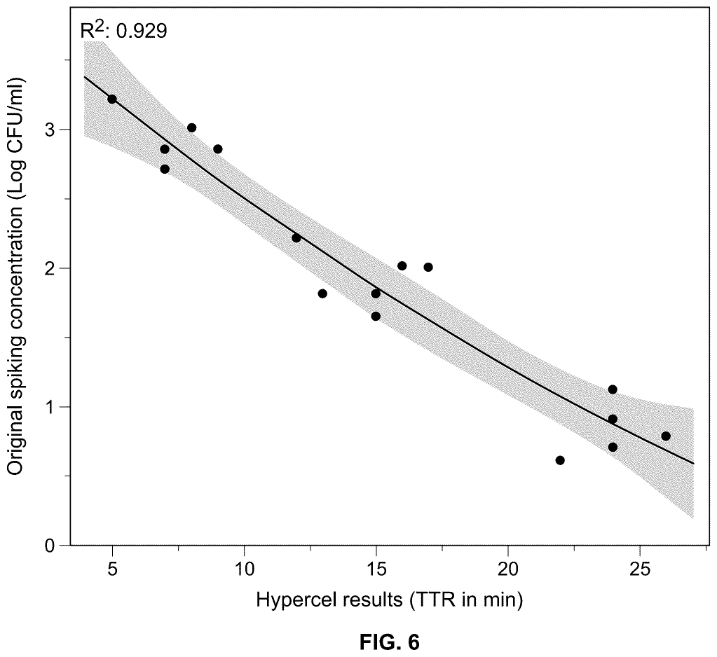

Hypercell Salmonella test provided robust semi-quantitative data (FIG. 6). Results obtained with Hypercell Salmonella test were within 0.69-log±0.4 agreement with the counts obtained with the microbiology plating method, across the three concentrations tested.

FIG. 6 depicts predicted CFU counts obtained with Hypercell Salmonella test versus confirmed counts obtained with microbiology method. Hypercell Salmonella counts were generated based on the linear correlation described in FIG. 5 Microbiology counts were recorded based on plating data.

### Example 5: Detection of Extremely Low Contaminant Levels with Rapid Enrichment

The ability of Hypercell tests to process large volumes of sample (up to 200 mL versus 1 mL for the standard methods) as seen above, provides the additional benefit of detecting the most dangerous pathogens (e.g., Salmonella, E. coli) at levels as low as 1 CFU with 3 hours of enrichment. By contrast, standard methods require a long period of enrichment (12-48 hours) to obtain the same level of sensitivity.

### Methodology

Liquid samples mimicking field conditions (10-200 mL final volume; i.e., MicroTally, Mitts) were inoculated with approximately one Salmonella cell per sample (according to the AOAC guidelines) and homogenized for 30 seconds. Inoculated samples were processed to detect Salmonella according to the Hypercell assay recommendations. Blank samples (n=9; sample before spiking with the microorganisms) were collected to assess the presence of pre-existing Salmonella in the samples, to avoid interference with the results obtained after spiking with a determined concentration of Salmonella. A total of 108 samples were processed for this study across three independent experiments and two operators.

Quantification of Salmonella spiking dose used for the liquid samples via microbiology plating method (XLT-4 agar); The same volume of inocula used for the spiking of the samples was plated on agar and inoculated plates were incubated at 35° C. for 24 hours before counting the colonies.

Detection of Salmonella post-enrichment using two-module filter assembly described in the present application: At a designated time point, up to 60 mL of enriched broth was processed using the wo-module filter assembly device. The final product extracted from the two-module filter assembly (50 μl) was plated on XLT-4 agar and inoculated plates were incubated at 35° C. for 24 hours before counting the colonies. A sample was considered as positive if at least 10 live Salmonella cells were recovered from the 50 μl of product extracted from the two-module filter assembly.

### Results

Two-module filter assembly reduces the required enrichment time for the detection of ultra-low concentration of contaminants (Table 5). The combination of enrichment and two-module filter assembly allows to detect c.a., 1 CFU per sample after a short enrichment (three hours), instead of the conventional method requiring 12-48 hours of enrichment.

TABLE 5
Bacterial recovery efficacy of ultra-low concentration
using two-module filter assembly described in the
present application after short enrichment.

| Salmonella | | Enrichment volume (ml) | | | | Blank |
|---|---|---|---|---|---|---|
| detection results* | | 10 | 50 | 100 | 200 | controls |
| Enrichment | 0 | 0% | 0% | 0% | 0% | 0% |
| time (hrs) | 1 | 25% | 0% | 0% | 0% | 0% |
| at 37° C. | 2 | 50% | 63% | 10% | 0% | 0% |
| | 3 | 100% | 100% | 75% | 5% | 0% |
| | 4 | 100% | 100% | 100% | 83% | 0% |
| | 5 | 100% | 100% | 100% | 100% | 0% |
| | 6 | 100% | 100% | 100% | 100% | 0% |
| | 7 | 100% | 100% | 100% | 100% | 0% |
| | 8 | 100% | 100% | 100% | 100% | 0% |

*Enriched samples were considered positive for Salmonella if at least 10 Salmonella cells were extracted using the two-module filter assembly device after a designated time post-enrichment. The percentage value indicates the proportion of positive enriched samples (at least 10 CFU were recovered using the two-module filter assembly device at the designated time point) compared to the proportion of negative enriched samples (Salmonella recovered after 24 hrs. enrichment but less than 10 CFU recovered at the designated time point).

### Example 6: Two-Module Filter Assembly Enables the Detection of Very Low Concentrations of Contaminants in Large Field Samples without Enrichment

Hypercell sample prep technology is able to detect very low concentrations of contaminants in large field samples in only a few minutes (liquid or solid suspended in liquid) maximizing detection sensitivity. This capability allows Hypercell to identify low concentrations of contaminants (<10 CFU/sample) in under 60 minutes without enrichment or ultra-low concentrations (1 CFU/sample) after a reduced enrichment period (three hours). By comparison, standard plating methods always require enrichment that takes from 12 hours to 48 hours.

### Methodology

Detection of low Salmonella concentration in large liquid samples without enrichment. Liquid samples (30-200 mL final volume) were inoculated with a known concentration of Salmonella (e.g., 1-70 CFU per sample) and homogenized for 30 seconds. Inoculated samples were processed to detect Salmonella according to Hypercell's assay recommendations. Blank samples (n=4; sample before spiking with the microorganisms) were collected to assess the presence of pre-existing Salmonella in the samples, to avoid interference with the results obtained after spiking with a determined concentration of Salmonella. A total of 74 samples were processed for this study by four operators.

Quantification of Salmonella spiking dose used for the liquid samples via microbiology plating method (XLT-4 agar); The same volume of inocula used for the spiking of the samples was plated on agar and inoculated plates were incubated at 35° C. for 24 hours before counting the colonies.

Recovery of Salmonella from liquid samples using Hypercell method: The product of the two-module filter assembly was plated on agar to demonstrate that Salmonella cells spiked in the large samples were properly concentrated and recovered. For this study, the bacterial counts recovered using the two-module filter assembly were compared with the bacterial counts obtained by plating the inocula, as described above following the microbiology method (XLT-4 agar). The following formula was used to determine the bacterial recovery rate of the two-module filter assembly: (counts obtained from Two-module filter assembly)/(counts obtained from inocula)*100.

### Results

Two-module filter assembly recovers small amounts (up to 2 cells) from large samples (up to 200 ml; Table 6). Within a few minutes, Salmonella was recovered from all spiked samples (n=16) independently of the sample volume (10-200 mL) and the spiking dose (2-100+CFU).

Two-module filter assembly provides robust and repeatable results across users (Table 7). Similar bacterial recovery rate was observed across the four operators.

Two-module filter assembly is a simple and accurate tool for quickly concentrating and extracting contaminants from field samples. No false results were detected using the Two-module filter assembly test.

TABLE 6
Bacterial recovery efficacy of Two-module filter assembly
based on inoculum dose and sample volume.

| | | | Bacterial | |
|---|---|---|---|---|
| | | | recovery | |
| Spiking dose | Sample volume | Sample size | efficacy | |
| (CFU/reaction) | (ml) | (n = 22) | (%) | |
| 30-70 | 30 | 5 | 100% | (5/5) |
| 10-30 | 30 | 5 | 100% | (5/5) |
| 3-6 | 30 | 5 | 100% | (5/5) |
| 2 | 30 | 5 | 100% | (5/5) |
| 2 | 200 | 2 | 100% | (2/2) |
| 0 | 30-200 | 6 | 0% | (0/6) |

TABLE 7
Bacterial recovery efficacy of Two-module
filter assembly between users.

| | | Bacterial | |
|---|---|---|---|
| | | recovery | |
| Spiking dose | Sample size | efficacy | Operator |
| (CFU/reaction) | (n = 52) | (% ± SD) | ID |
| 13 | 12 | 98 ± 21 | User 1 |
| 20 | 12 | 104 ± 7 | User 2 |
| 40 | 12 | 99 ± 4 | User 3 |
| 74 | 4 | 93 ± 5 | User 4 |
| 212 | 12 | 100 ± 1 | User 1 |
| 0 | 4 | 0 | User 1-4 |

### Example 7: Hypercell Technology is Compatible with a Broad Variety of Field Samples

The tests provide the same level of accuracy and ease of use across a wide range of sample types (food, surfaces, liquids, air), making Hypercell tests adaptable to various testing needs.

### Methodology

Hypercell is designed for broad compatibility across a wide range of sample types, making it a versatile solution for food safety testing. Its robust detection system seamlessly integrates with in-process samples, including swabs, sponges, MicroTally/Mitts, poultry carcass rinses, fermenter broth products, and water wash tanks, among others, ensuring reliable monitoring throughout production. Additionally, Hypercell is well-suited for final product testing, accurately detecting pathogens in chicken breast, ground beef, meat trims, yeast products, and fresh produce. This flexibility enables food producers to implement a single, highly-efficient testing platform across multiple stages of processing, enhancing safety and compliance with industry regulations.

### Results

Table 8 provides a recap of the different field samples successfully processed with Hypercell technology for the detection of specific contaminants (e.g., Salmonella, Listeria, EB, E. coli). Results obtained with the Hypercell tests were compared to USDA standard detection protocols. Since October 2024, over 150 field samples have been processed for the validation of Hypercell tests against various matrices (i.e., food, swabs, liquid). Overall, these validations demonstrated that Hypercell technology detected down to 10 CFU under 60 minutes when a sample input size of 3, 5 or 10 mL was used.

TABLE 8
Variety of samples processed using Hypercell technology.

| Sample | | Recommended |
|---|---|---|
| categories | Sample types | input size |
| In process | Small environmental swab (swab, | All the recovered |
| samples | sponge) | liquid |
| | Large environmental swab (MicroTally, | Up to 10 ml |
| | Mitts) | |
| | Poultry carcass Rinse | Up to 10 ml |
| | Fermenter broth product | Up to 10 ml |
| | Water wash tank | Up to 10 ml |
| Final | Meat samples (beef, pork, poultry) | Up to 10 ml |
| product | Yeast product | Up to 10 ml |
| samples | Fresh produce | Up to 10 ml |

### Example 8: High Specificity for Targeted Bacterial Detection

Hypercell tests are designed to identify the most frequent strains of a specific microorganisms without identifying other microorganisms avoiding false positive reactions (identification of the wrong microorganisms) and false negatives (missing some strains of the same microorganisms). Going a step further in the specificity scale, Hypercell tests can also effectively distinguish pathogenic from non-pathogenic species within the same taxonomic classification (e.g., non-pathogenic E. coli vs E. coli O157, non-pathogenic Listeria vs Listeria monocytogenes), reducing false positives that could lead to unnecessary interventions and costly delays. This unique feature allows food producers to assess risk more precisely and make more informed decisions.

To validate these features, a comprehensive microorganism library consisting of over 500 strains is utilized to conduct specificity testing. This extensive collection enables the evaluation of assays against a diverse range of target and non-target microorganisms, ensuring precise pathogen detection with minimal cross-reactivity.

### Methodology

The specificity of Hypercell tests were validated in accordance with AOAC inclusive/exclusive testing standards. The inclusive and exclusive lists are essential for specificity testing, ensuring that Hypercell tests accurately detect target microorganisms while excluding non-targets.

The inclusive list (n>50) consists of diverse strains of the same target microorganism, verifying that the test consistently identifies all relevant variants (=no false negative).

The exclusive list (n>30) includes closely related but non-target microorganisms, ensuring no cross-reactivity (=no false positive).

To assess specificity, pure bacterial cultures were tested at both low (<100 CFU/reaction) and high (>105 CFU/reaction) concentrations using the Hypercell tests.

A positive result was defined by the detection of a fluorescent signal exhibiting at least a 100-fold increase over background noise (blank sample) within the optimal incubation period, which allows for the detection of as few as 10 CFU of the target microorganism in less than 60 minutes.

A negative result was recorded if no fluorescent signal was observed within this timeframe.

### Results

Robust detection of all regulated pathogenic bacterial strains:

Hypercell O157, Hypercell Salmonella and Hypercell LM tests detect with high specificity all targeted microorganisms (E. coli O157 strains, Salmonella spp. and Listeria monocytogenes serogroups, respectively), preventing the risk of false negative results.

Robust detection of key microbial population (pathogenic versus non-pathogenic forms)

Hypercell Listeria test enables the rapid and highly accurate detection of all Listeria species, except L. innocua, achieving a sensitivity of <10 CFU in under 60 minutes. L. innocua, a non-pathogenic strain that shares ecological niches with L. monocytogenes, is a common source of false-positive results in traditional testing methods. These false positives can trigger unnecessary food safety interventions, leading to costly disruptions. Hypercell's unique ability to exclude L. innocua while reliably detecting pathogenic Listeria species will improve risk assessment and enable more effective food safety decisions.

Hypercell EB Core and Hypercell EB tests: Hypercell engineered very specific Enterobacteriaceae detection tests to rapidly detect the most dangerous microorganisms without getting positive results from less relevant microorganisms. This advanced detection capability allows food producers to take swift action to protect public health and maintain regulatory compliance. This test will be proposed along with the more traditional EB test that identifies the same Enterobacteriaceae as the current standard method.

Hypercell ST-EC test enables rapid detection of Shiga-toxin producing E. coli.

TABLE 9
Specificity detection testing of Hypercell tests against inclusive
and exclusive lists (n = 203 microorganisms total).

| | | | Hypercell tests | | | | | | |
|---|---|---|---|---|---|---|---|---|---|
| | | Number | | EB | | | | | |
| | | of | | Core | ST- | | | | |
| Category | Name | strains | EB | EZ | EC | O157 | Salmonella | Listeria | LM |
| Enterobacteriaceae | Citrobacter | 1 | 100% | 0 | 0 | 0 | 0 | 0 | 0 |
| | Enterobacter | 3 | 100% | 0 | 0 | 0 | 0 | 0 | 0 |
| | Escherichia coli | 2 | 100% | 100% | 0 | 0 | 0 | 0 | 0 |
| | (non-pathogenic) | | | | | | | | |
| | Escherichia coli (ST, | 18 | 100% | 100% | 100% | 0 | 0 | 0 | 0 |
| | UPEC, UTI, ETEC) | | | | | | | | |
| | Escherichia coli | 50 | 100% | 100% | 100% | 100% | 0 | 0 | 0 |
| | O157 | | | | | | | | |
| | Hafnia | 1 | 100% | 0 | 0 | 0 | 0 | 0 | 0 |
| | Klebsiella | 4 | 100% | 0 | 0 | 0 | 0 | 0 | 0 |
| | Pantoea | 1 | 100% | 0 | 0 | 0 | 0 | 0 | 0 |
| | Proteus | 1 | 100% | 0 | 0 | 0 | 0 | 0 | 0 |
| | Salmonella | 48 | 100% | 100% | 0 | 0 | 100% | 0 | 0 |
| | Shewanella | 1 | 100% | 0 | 0 | 0 | 0 | 0 | 0 |
| | Shigella | 1 | 100% | 0 | 0 | 0 | 0 | 0 | 0 |
| | Yersinia | 1 | 100% | 100% | 0 | 0 | 0 | 0 | 0 |
| | Pseudomonas | 3 | 0 | 0 | 0 | 0 | 0 | 0 | 0 |
| | Saccharomyces | 6 | 0 | 0 | 0 | 0 | 0 | 0 | 0 |
| | Staphylococcus | 2 | 0 | 0 | 0 | 0 | 0 | 0 | 0 |
| | Xanthomonas | 2 | 0 | 0 | 0 | 0 | 0 | 0 | 0 |
| Listeria | Listeria (non-LM) | 6 | 0 | 0 | 0 | 0 | 0 | 100% | 0 |
| | Listeria | 40 | 0 | 0 | 0 | 0 | 0 | 100% | 100% |
| | monocytogenes | | | | | | | | |
| | (LM) | | | | | | | | |
| Other | Acidovorax | 1 | 0 | 0 | 0 | 0 | 0 | 0 | 0 |
| microorganisms | Bacillus | 3 | 0 | 0 | 0 | 0 | 0 | 0 | 0 |
| | Buttiauxella | 1 | 0 | 0 | 0 | 0 | 0 | 0 | 0 |
| | Clavibacter | 1 | 0 | 0 | 0 | 0 | 0 | 0 | 0 |
| | Clostridium | 1 | 0 | 0 | 0 | 0 | 0 | 0 | 0 |
| | Flavobacterium | 1 | 0 | 0 | 0 | 0 | 0 | 0 | 0 |
| | Lactobacillus | 3 | 0 | 0 | 0 | 0 | 0 | 0 | 0 |
| | Macrococcus | 1 | 0 | 0 | 0 | 0 | 0 | 0 | 0 |
| | Pseudomonas | 3 | 0 | 0 | 0 | 0 | 0 | 0 | 0 |
| | Saccharomyces | 6 | 0 | 0 | 0 | 0 | 0 | 0 | 0 |
| | Staphylococcus | 2 | 0 | 0 | 0 | 0 | 0 | 0 | 0 |
| | Xanthomonas | 2 | 0 | 0 | 0 | 0 | 0 | 0 | 0 |

Cells indicate a positive (white) or negative (grey) detection of the designated microorganism (row) with the corresponding Hypercell (column), respectively. “100%” indicates that all strains were detected (pure bacterial cultures were tested at both low (<100 CFU/reaction) and high (>10⁵ CFU/reaction) concentrations using the Hypercell tests).

### Example 9: Isothermal Technology Enables Pathogen Detection Using Multiple Fluorescent Thermocyclers

The isothermal detection system described herein demonstrated excellent compatibility across three distinct fluorescence detection platforms: Aladdin Analyzer™ (Hypercell), Mini 8-Hole Isothermal Fluorescence PCR (Laboao), and CFX96 Touch Real-Time PCR Detection System (Bio-Rad). Consistent fluorescence signal profiles and time-to-result performance were observed across all platforms, with no false positives or false negatives recorded. This confirms the flexibility and reliability of the Hypercell assay across a range of operational settings.

### Materials and Methods

Fluorescent Detection Platforms Evaluated: detection platforms were evaluated: 1) Aladdin Analyzer (Hypercell): A compact, portable, and cost-effective fluorescence reader optimized for Hypercell isothermal chemistry. Delivers rapid, high-sensitivity detection in less than 60 minutes with minimal user input. 2) Mini 8-Hole Isothermal Fluorescence PCR (Laboao): A low-cost benchtop instrument designed for field or small-lab applications. Supports isothermal amplification and real-time fluorescence detection for up to 8 samples using FAM-compatible probes. 3) CFX96 Touch Real-Time PCR Detection System (Bio-Rad): A high-throughput, research-grade qPCR system widely used in academic and industrial labs. Compatible with the assays disclosed herein using FAM channels and standard 96-well plate formats

### Experimental Design

Pathogen Target: Salmonella enterica

Positive samples (n=5 per unit): tubes of Hypercell lyophilized isothermal master mix were rehydrated with 50 μL of elution buffer spiked with approximately 500 CFU of Salmonella.

Negative controls (n=5 per unit): tubes were rehydrated with 50 μL of elution buffer without Salmonella.

Incubation: All tubes were run at 65° C. for 60 minutes. Fluorescence (excitation 495 nm) was recorded every 20 seconds.

Total reactions: 30 reactions used in this study (15 positive, 15 negative)

### Results

Salmonella was consistently detected across all three devices with an average time to detection of 13±2.2 minutes. No fluorescence signal was detected in any of the negative control reactions over the 60-minute run time. No false positives or false negatives were recorded, confirming the high specificity and sensitivity of the detection chemistry disclosed herein regardless of the fluorescence reader used.

TABLE 10
Detection speed and accuracy of technology
across three fluorescent units.

| | Fluorescent units | | |
|---|---|---|---|
| | CFX96 | Aladdin | Mini 8- |
| Fluorescence signal | Touch | Analyzer | Hole PCR |
| detection (time to | (Bio-Rad; | (Hypercell; | (Laboao; |
| result in min) | n = 5 reps) | n = 5 reps) | n = 5 reps) |
| Spiked (500 cells) | 13 ± 2.2 | 13 ± 1.3 | 13 ± 1.8 |
| Not spiked (negative) | ND | ND | ND |

ND: No fluorescent signal detected within 60 minutes at 65° C.

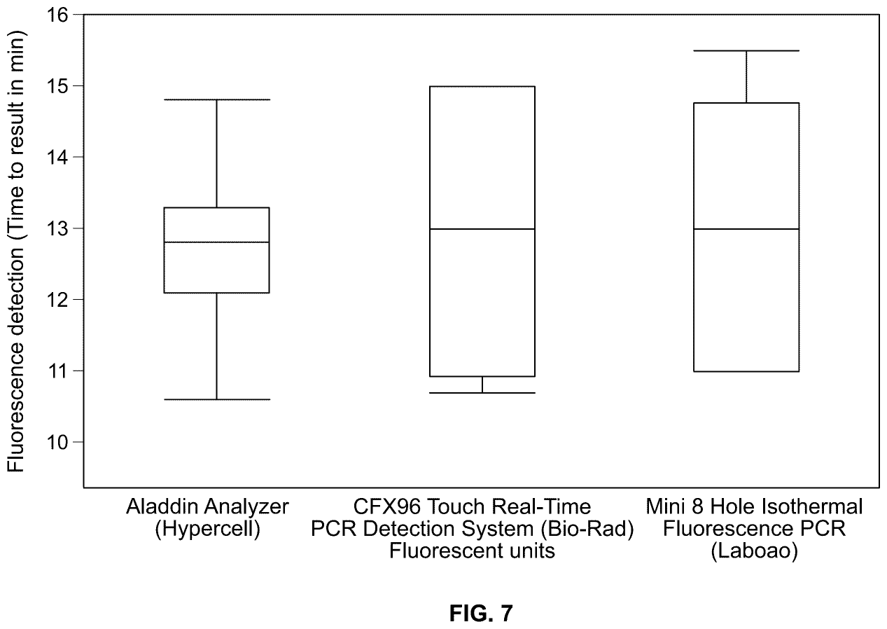

FIG. 7 depicts the detection profiles across fluorescence platforms. It shows head-to-head comparison of Hypercell isothermal detection on three commercial fluorescence readers. All reactions were spiked with ˜500 CFU of Salmonella (positive). N=5 reps per unit.

What is claimed is:

1. A filtration system for capturing targeted microobjects from a sample, the system comprising:
a) a first filtration module configured to capture particles at least twice as large as the targeted microobjects;
b) a second filtration module downstream of and in fluid communication with the first filtration module, configured to capture the targeted microobjects from the sample;
c) an injector for injecting the sample, configured to be connected upstream and in fluid communication with the first filtration module; and
d) a second injector configured to be connected upstream of and in fluid communication with the first filtration module,
wherein the second injector is configured to resuspend the captured microobjects in a resuspension solution, and
wherein the resuspension solution comprises a buffer solution.

2. The system of claim 1, further comprising a flow regulator in fluid connection with and configured between the first filtration module and the second filtration module, wherein flow regulator comprises:
a) an upper connector configured to connect the flow regulator to the first filtration module;
b) a lower connector configured to connect the flow regulator to the second filtration module;
c) a side channel connector configured to connect the second injector to the flow regulator; and
d) a 3-way valve configured to change orientation to direct flow independently, the 3-way valve having at least a first flow orientation and a second flow orientation,
wherein the first flow orientation is configured to seal the side channel connector and produce fluid communication between the upper connector and the lower connector,
wherein the second flow orientation is configured to seal the upper connector and produce fluid communication between the side channel connector and the lower connector, and
wherein the orientation of the 3-way valve is controlled by a rotating handle.

3. The system of claim 2, further comprising a cylindrical shell comprising a top end, a bottom end, and an outer wall, configured to encompass the first filtration module, flow regulator, and second filtration module, the cylindrical shell comprising:
a) a first injector opening in the top end configured to provide access to the first filtration module;
b) a second injector opening in the outer wall which provides access to the side channel connector;
c) a turning knob opening in the outer wall comprising a turning knob operably connected with the rotating handle, wherein the turning knob opening is configured to change the orientation of the 3-way valve; and
d) a waste chamber, wherein the waste chamber is located downstream of the second filtration module and within the shell;
wherein the shell is composed of materials selected from the group comprising polylactic acid (PLA), polyethylene terephthalate glycol (PETG), thermoplastic polyurethane (TPU), nylon, or other 3D printed filaments, wood- or water-based filaments, acrylonitrile butadiene styrene (ABS), polypropylene (PP), polyethylene (PE) polystyrene (PS), polycarbonate (PC), or other plastic injection molding polymers, polypropylene resin, waterproof paper tubing, including 2 mm cardboard covered with 157G coating, and aluminum, and
wherein the shell protects the encompassed items and prevents spilling.

4. The system of claim 1, wherein the first filtration module further comprises a filter cartridge encompassing one or more layers of one or more filters selected from a group comprising polyester mesh, nitrocellulose, nylon, cellulose acetate, polyether sulphone (PES), cellulose nitrate (collodion), cellulose acetate (CA), mixed cellulose esters (MCE), other cellulose membrane filters, polycarbonate, polyethylene terephthalate (PET), polyvinylidene fluoride (PVDF), ceramic membranes, polypropylene (PP), polycarbonate (PC), polytetrafluoroethylene (PTFE), and polyester (PETE), and wherein the one or more filters has a pore diameter between 0.22 and 300 μm and a filter diameter between 4 and 33 mm.

5. The system of claim 1, wherein the second filtration module further comprises a filter cartridge encompassing one or more layers of one or more filter membranes selected from the list comprising nitrocellulose, nylon, cellulose acetate, polyether sulphone (PES), cellulose nitrate (collodion), other cellulose membrane filters, polycarbonate, polyethylene terephthalate (PET), polyvinylidene fluoride (PVDF), ceramic membranes, polypropylene (PP), polytetrafluoroethylene (PTFE), and polyester (PETE), and wherein the one or more filter membranes has a pore diameter between 0.05 and 20 μm and a filter diameter between 4 and 33 mm.

6. The system of claim 1, wherein the injector is selected from the group comprising a syringe; an air line; a fluid line comprising one or more of irrigation water, fermentation input including feed lines and buffers, and outputs, including harvest outputs; a line with a positive displacement pump, comprising reciprocating pumps, rotary pumps, and peristaltic pumps; a line with any centrifugal pump, comprising radial flow pumps, axial flow pumps, and mixed flow pumps; and a line with a pump selected from a group comprising submersible pumps, jet pumps, hand pumps, sump pumps, and sewage pumps.

7. The system of claim 3, wherein the waste chamber contains pre-loaded absorbent material capable of absorbing liquid to transform liquid waste into semi-solid waste to prevent leakage, the pre-loaded absorbent material selected from a list comprising viscose, cotton, bamboo fiber, and polyester.

8. The system of claim 7, wherein the pre-loaded absorbent material is pre-treated with antimicrobial material selected from the list comprising silver ions, benzalkonium chloride, chlorhexidine, and essential oil.

9. The system of claim 1, wherein the second filtration module further comprises an isolated chamber comprising dried isothermal reagents and primers configured to release the dried isothermal reagents and primers into the second filtration module.

10. The system of claim 1, wherein the buffer solution comprises water, 0.1-100 mM Tris, 0.1-100 mM EDTA, 0.005-0.02% trehalose, and 0.002-2% tween.

11. The system of claim 1, wherein the injector further comprises an injector adapter fitted in and removable from the injector and one or more pre-loaded solvents, wherein the injector adapter is a conical tube comprising:
a) an upper opening configured to accept the sample, wherein the sample is a swab;
b) a lower opening;
c) an outer surface comprising an O-ring configured to prevent leaking between the outer surface and the injector;
d) an inner surface comprising a plurality of teeth configured to agitate the swab to suspend the targeted microobjects; and
e) one or more aeration holes through the conical tube configured to optimize liquid and air transfer to prevent pressurization and depressurization of the adapter;
wherein the one or more pre-loaded solvents is selected from a list comprising buffered peptone water, phosphate buffered salt, neutralized buffered peptone water (nBPW), Letheen broth, universal transport media (UTM), Nutrient Broth (NB), Luria Bertani (LB), Tryptic Soy Broth (TSB), Brain Heart Infusion broth (BHI), and water.

12. An automated processing system comprising:
a) one or more processing modules, each comprising:
i) the system of claim 2, wherein the first filtration module further comprises an input 3-way flow regulator connected to the first filtration module, and wherein the injector is a fluid line;
ii) a collection tube containing a sample, wherein the collection tube and the input 3-way flow regulator are in fluid communication by the fluid line;
iii) a top motor configured to control the orientation of the input 3-way flow regulator;
iv) a bottom motor configured to control the orientation of the flow regulator;
b) an elution reservoir containing a buffer solution, wherein the elution reservoir is in fluid communication with the input 3-way flow regulators of the one or more processing modules;
c) a module frame containing the one or more processing modules; and
d) a waste collection tank configured downstream of the system of the one or more processing modules.

13. A sampling bag configured to prepare a sample for injection into the filtration system of claim 1, the sampling bag comprising:
a) a flexible front and rear wall, the walls being joined together along two side edges and along a bottom edge extending between the side edges to define an internal cavity with a top opening;
b) a deformable wire connected to a wall of the bag below the top opening, comprising ends extending laterally from each side of the bag, the ends of the deformable wire being adapted to be bent inwardly to close the top opening;
c) an opening in the front wall comprising a removable cap configured to seal the opening and provide access to the internal cavity by the injector of claim 1 configured to eject and withdraw liquid;
d) a filter configured to remove large particles prior to withdrawal of liquid from the opening; and
wherein the sampling bag is configured to receive a sample and allows for easy preparation of the sample.

14. A method for detecting microorganisms in a sample comprising:
a) concentrating microorganisms from the sample using the system of claim 1, the steps comprising:
i) loading the sample into the injector;
ii) injecting the sample into the system;
iii) capturing microorganisms from the sample in the second filtration module;
iv) resuspending the captured microorganisms in a resuspension solvent, producing a concentrated sample;
v) withdrawing the concentrated sample by the second injector;
b) transporting the concentrated sample to a microanalyzer; and
c) detecting the microorganisms in the concentrated sample by the microanalyzer.

15. The method of claim 14, wherein the method further comprises releasing pre-loaded dried isothermal reagents and primers into the second filtration module after resuspension of the captured microorganisms.

16. A point-of-use system for detecting pathogenic microorganisms, comprising the system of claim 1 and a microanalyzer, wherein the system allows for filtration of a sample, capture of microorganisms, and analysis of microorganisms captured.

17. The point-of-use system of claim 16 wherein the microanalyzer is selected from a group comprising an ALADDIN ANALYZER™, an EzDx WeD-1 Pro device, a Mini 8-Hole Isothermal Fluorescence PCR, a CFX96 Touch Real-Time PCR Detection System.

18. A kit for the detection of pathogenic microorganisms, comprising:
a) the system of claim 3;
b) a sterile PCR tube preloaded with isothermal reagents; and
c) an apparatus for testing DNA.

19. The kit of claim 18, further comprising a swab for solid samples and an injector adapter fitted in and removable from the injector and one or more pre-loaded solvents, wherein the injector adapter is a conical tube comprising:
a) an upper opening configured to accept the sample, wherein the sample is a swab;
b) a lower opening;
c) an outer surface comprising an O-ring configured to prevent leaking between the outer surface and the injector;
d) an inner surface comprising a plurality of teeth configured to agitate the swab to suspend the targeted microobjects; and
e) one or more aeration holes through the conical tube configured to optimize liquid and air transfer to prevent pressurization and depressurization of the adapter;
wherein the one or more pre-loaded solvents is selected from a list comprising buffered peptone water, phosphate buffered salt, neutralized buffered peptone water (nBPW), Letheen broth, universal transport media (UTM), Nutrient Broth (NB), Luria Bertani (LB), Tryptic Soy Broth (TSB), Brain Heart Infusion broth (BHI), and water.

20. The kit of claim 18, further comprising a sampling bag configured to prepare a sample for injection into the filtration system of claim 1, the sampling bag comprising:
a) a flexible front and rear wall, the walls being joined together along two side edges and along a bottom edge extending between the side edges to define an internal cavity with a top opening;
b) a deformable wire connected to a wall of the bag below the top opening, comprising ends extending laterally from each side of the bag, the ends of the deformable wire being adapted to be bent inwardly to close the top opening;
c) an opening in the front wall comprising a removable cap configured to seal the opening and provide access to the internal cavity by the injector of claim 1 configured to eject and withdraw liquid; and
d) a filter configured to remove large particles prior to withdrawal of liquid from the opening;
wherein the sampling bag is configured to receive a sample and allows for easy preparation of the sample.
